SEQUENCE LISTING (11 sequences)

SEQ ID NO: 1 - DNA, 20 bp, synthetic construct
  misc_feature 1..20: note = Forward primerfor the pGL4.31
  source 1..20: mol_type = other DNA
ttccggagtactgtcctccg

SEQ ID NO: 2 - DNA, 20 bp, synthetic construct
  misc_feature 1..20: note = Reverse primer for the pGL4.31
  source 1..20: mol_type = other DNA
ggtagaatggcgctgggccc

SEQ ID NO: 3 - DNA, 21 bp, synthetic construct
  misc_feature 1..21: note = Distal forward primer for p21 promoter:
  source 1..21: mol_type = other DNA
tgcttcccaggaacatgcttg

SEQ ID NO: 4 - DNA, 21 bp, synthetic construct
  misc_feature 1..21: note = Distal reverse primer for p21 promoter
  source 1..21: mol_type = other DNA
ctgaaaacaggcagcccaagg

SEQ ID NO: 5 - DNA, 20 bp, synthetic construct
  misc_feature 1..20: note = Proxima forward primer for p21 promoter
  source 1..20: mol_type = other DNA
gcagaggagaaagaagcctg

SEQ ID NO: 6 - DNA, 20 bp, synthetic construct
  misc_feature 1..20: note = Proxima reverse primer for p21 promoter
  source 1..20: mol_type = other DNA
gcagaggagaaagaagcctg

SEQ ID NO: 7 - DNA, 20 bp, synthetic construct
  misc_feature 1..20: note = p21 (transcription start site)forward primer
  source 1..20: mol_type = other DNA
gcagaggagaaagaagcctg

SEQ ID NO: 8 - DNA, 18 bp, synthetic construct
  misc_feature 1..18: note = p21 (transcription start site) reverse primer
  source 1..18: mol_type = other DNA
gctctctcacctcctctg

SEQ ID NO: 9 - DNA, 20 bp, synthetic construct
  misc_feature 1..20: note = GAPDHTSS forward primer
  source 1..20: mol_type = other DNA
ggctcccacctttctcatcc

SEQ ID NO: 10 - DNA, 20 bp, synthetic construct
  misc_feature 1..20: note = GAPDH TSS reverse primer
  source 1..20: mol_type = other DNA
ggccatccacagtcttctgg

SEQ ID NO: 11 - protein, 23 aa, synthetic construct
  REGION 1..23: note = Peptidederived from human histone H4
SGRGKGGKGLGKGGAKRHRKVLR

(19) United States
(12) Patent Application Publication
(10) Pub. No.: US 2025/0134952 A1
(43) Pub. Date: May 1, 2025

(54) METHODS FOR IMPROVING THE EFFICACY OF HDAC INHIBITOR THERAPY AND PREDICTING THE RESPONSE TO TREATMENT WITH HDAC INHIBITOR

(71) Applicants: INSTITUT NATIONAL DE LA SANTÉ ET DE LA RECHERCHE MÉDICALE, Paris (FR); CENTRE HOSPITALIER REGIONAL UNIVERSITAIRE DE LILLE, Lille (FR); UNIVERSITÉ DE LILLE, Lille (FR)

(72) Inventors: Luc BUEE, Lille Cedex (FR); Bruno Georges LEFEBVRE, Lille Cedex (FR); Thomas RICO, Grand-Failly (FR)

(21) Appl. No.: 18/692,432

(22) PCT Filed: Sep. 20, 2022
(86) PCT No.: PCT/EP2022/076112

§ 371 Date: Mar. 15, 2024

(30) Foreign Application Priority Data
Sep. 20, 2021 (EP) 21306299.5
Dec. 22, 2021 (EP) 21306903.2

(51) Int. Cl. A61K 38/15; A61K 31/167; A61K 31/4045; A61K 31/4406; A61K 31/506; A61P 35/00; C07K 16/18; G01N 33/68

(52) U.S. Cl. CPC A61K 38/15; A61K 31/167; A61K 31/4045; A61K 31/4406; A61K 31/506; A61P 35/00; C07K 16/18; G01N 33/6893; G01N 2333/46

(57) ABSTRACT

Investigating the impact of Tau protein expression in cancer cell lines, Inventors have demonstrated that the Tau expression is associated with an increased resistance to HDAC inhibitors. Briefly in the present invention, inventors report that Tau expression in breast cancer cell lines causes resistance to the anti-cancer effects of histone deacetylase inhibitors, by preventing histone deacetylase inhibitor-inducible gene expression and remodeling of chromatin structure. Inventors identify Tau as a protein recognizing and binding to core histone when H3 and H4 are devoid of any post-translational modifications or acetylated H4 that increases the Tau's affinity. In addition, they demonstrate that the interaction between Tau and histones prevents further histone H3 post-translational modifications induced by histone deacetylase-inhibitor treatment by maintaining a more compact chromatin structure The present invention relates to means to improve the bioavailability of histone deacetylase (HDAC) inhibitor and thereby also improve the efficacy of histone deacetylase (HDAC) inhibitor treatments.

### FIELD OF THE INVENTION

The present invention is in the field of medicine, in particular oncology, for improving the efficacy of histone deacetylase (HDAC) inhibitor therapies and for predicting the response to treatment with HDAC inhibitor.

### BACKGROUND OF THE INVENTION

Tau was first described as a neuronal microtubule-associated protein (MAP), regulating microtubule assembly and axonal transport. In the brain, the Microtubule Associated protein Tau (MAPT) constitutes a family of six isoforms containing three or four microtubule binding domains (named 3R and 4R respectively). The 4R isoforms have a higher affinity for microtubules (Wang and Mandelkow, 2016). In mouse, Tau3R(s) are expressed mostly during development whereas Tau4R becomes the predominant isoform in adult brain. It is thought that the lower affinity of Tau3R for microtubules allows the morphological changes necessary for neuronal differentiation and migration (Lu and Kosik, 2001; Avila et al., 2004; Sergeant et al., 2005). The affinity of Tau for microtubules is also tightly regulated by post-translational modifications. In tauopathies, there is abnormal Tau phosphorylation, leading it to detach from microtubules and favoring aggregation. In addition, several mutations in the MAPT gene have been identified in the inherited frontotemporal dementia and parkinsonism linked to chromosome 17 (FTDP-MAPT). Known mutations either reduce Tau affinity for microtubules or change the ratio (3R/4R) (Goedert and Spillantini, 2000).

Since its original discovery as a brain disease gene, Tau expression has been detected in several non-neuronal cells like kidney, liver and muscle. Furthermore, Tau is overexpressed in different human breast, gastric, prostate cancer cell lines and tissues (Gargini et al., 2019). Previous studies have suggested that Tau expression could be a predictive marker for paclitaxel resistance in different cancer types (Wagner et al., 2005; Lei et al., 2020). At the molecular level, it has been demonstrated that Tau protects microtubule from paclitaxel binding by binding to Tubulin (Smoter et al., 2011).

However, recent studies show that the role of Tau is not limited to microtubule dynamics. It has been demonstrated that Tau interferes with several biological processes such as signaling pathways, synaptic functions, RNA metabolism, and DNA integrity and it even contributes to the inflammatory response (Bou Samra et al., 2017; Lebouvier et al., 2017; Sotiropoulos et al., 2017). Most intriguingly, chromatin abnormalities were detected in neurons from several tauopathy models, as well as in human pathological brains. Indeed, there are large-scale changes in histone acetylation, throughout the epigenome in Tau pathologies (Klein et al., 2019). Supporting the likely importance of Tau's effect on chromatin, overexpression of a FTDP-MAPT mutant in drosophila led to global chromatin relaxation, showed by the loss of H3K9me2 and altered distribution of heterochromatin-associated protein HP1 (Frost et al., 2014). In addition, peripheral cells from patients carrying Tau mutations also displayed chromosome numerical and structural aberrations as well as chromatin anomalies (Rossi et al., 2018). Frost et al. further demonstrated that this heterochromatin relaxation, thought to be a consequence of DNA damage induced by oxidative stress, allowed the expression of genes normally repressed in heterochromatin. Although the heterochromatin maintenance has been attributed to nuclear Tau in this context, the molecular mechanisms remain elusive (Mansuroglu et al., 2016).

In the past decades, several histone deacetylase-inhibitors have been developed (Li and Seto, 2016). At the molecular level, these compounds lead to accumulation of acetylated histones and non-histone proteins such as transcription factors, tubulin and heat-shock proteins, selectively altering gene expression (Glaser et al., 2003; Mitsiades et al., 2004). Global changes in chromatin supra-organization due to histone deacetylase-inhibitors can be also observed using different techniques such confocal laser scanning microscopy or enhanced sensitivity of DNA to nucleases (Toth et al., 2004; Gorisch et al., 2005; Marchion et al., 2005; Bustos et al., 2017). In addition, previous studies show that upon inhibition of acetylation, heterochromatin binding proteins reversibly detach and disperse within the nucleus (Taddei et al., 2001; Robbins et al., 2005; Cowell et al., 2011). The loss of binding of HP1s to heterochromatin is also thought to be important in the histone deacetylase-inhibitor mechanism of action as it induces also abnormal mitosis (Morgan and Shilatifard, 2015).

To dissect the role of Tau in controlling chromatin functions and/or organization, we sought to perturb chromatin structure by inhibiting histone deacetylation with the pan-histone deacetylase-inhibitor trichostatin A (TSA). Here we described Tau as a new histone binding protein that stabilized condensed chromatin under histone deacetylase-inhibitor treatment, in part by preventing post-translational modification of histones. Taken together, our results shed new light on the role of Tau on chromatin organization in neuronal and non-neuronal cells.

### SUMMARY OF THE INVENTION

The present invention relates to means to predict the response to histone deacetylase (HDAC) inhibitor and thereby also to improve the efficacity of histone deacetylase (HDAC) inhibitor treatments.

In a first aspect, the present invention relates to a method for treating cancer a patient undergoing HDAC inhibitor therapy comprising the step of
determining in a biological sample obtained from said patient the level of Tau expression and
administering a therapeutically effective amount of HDAC inhibitor when the level of Tau expression is low or null.

In a second aspect the invention relates to a method of preventing emergence of resistance to treatment with a HDAC inhibitor in a subject in need thereof comprising administering to the subject, a Tau inhibitor

In a third aspect, the invention relates to a method for preventing and/or treating cancer with acquired resistance to treatment with a HDAC inhibitor in a subject in need thereof comprising administering to the subject a combination of drugs selected from the group consisting of HDAC inhibitor and a Tau inhibitor.

In another aspect, the present invention relates to a method for predicting the response to a histone deacetylase (HDAC) inhibitor treatment in a patient suffering from a cancer, comprising the step of determining in a biological sample obtained from said patient the level of Tau protein expression, wherein the level of Tau protein expression is predictive of a response to a HDAC inhibitor (HDACi) treatment.

### DETAILED DESCRIPTION OF THE INVENTION

Investigating the impact of Tau protein expression in cancer cell lines, the Inventors have demonstrated that the Tau expression is associated with an increased resistance to HDAC inhibitors. Briefly in the present invention, inventors report that Tau expression in breast cancer cell lines causes resistance to the anti-cancer effects of histone deacetylase inhibitors, by preventing histone deacetylase inhibitor-inducible gene expression and remodeling of chromatin structure. Inventors identify Tau as a protein recognizing and binding to core histone when H3 and H4 are devoid of any post-translational modifications or acetylated H4 that increases the Tau's affinity. Consistent with chromatin structure alterations in neurons found in frontotemporal lobar degeneration, Tau mutations did not prevent histone deacetylase-inhibitor-induced higher chromatin structure remodeling by suppressing Tau binding to histones. In addition, they demonstrate that the interaction between Tau and histones prevents further histone H3 post-translational modifications induced by histone deacetylase-inhibitor treatment by maintaining a more compact chromatin structure

Altogether, these results highlight a new cellular role for Tau as a chromatin reader, which paves the way to the development of a personalized treatment for cancer patients with HDAC inhibitor.

### Therapeutic Method

A first aspect of the present invention relates to a method of treating cancer in a patient undergoing HDAC inhibitor therapy, comprising the step of:
Determining in a biological sample obtained from said patient the level of Tau expression,
Administering a therapeutically effective amount of HDAC inhibitor to the patient when the level of Tau expression is low or null.

In a second aspect, the invention relates to a method of preventing emergence of resistance to treatment with a HDAC inhibitor in a subject in need thereof comprising administering to the subject, a Tau inhibitor

In third aspect, the invention relates to a method for preventing and/or treating cancer with acquired resistance to treatment with a HDAC inhibitor in a subject in need thereof comprising administering to the subject a combination of drugs selected from the group consisting of HDAC inhibitor and an Tau inhibitor.

As used herein, the term “patient” denotes a mammal, such as a rodent, a feline, a canine, and a primate. Preferably, a patient according to the invention is a human. Typically, a patient according to the invention refers to any subject (preferably human) afflicted with or susceptible to be afflicted with a cancer.

As used herein, the terms “cancer” and “tumors” refer to or describe the pathological condition in mammals that is typically characterized by unregulated cell growth. More precisely, in the methods of the invention, diseases, namely tumors that not express/secrete Tau protein are most likely to respond to the HDAC inhibitor treatment, or after using a Tau inhibitor. In particular, the cancer may be associated with a solid tumor or lymphoma/leukemia (from hematopoietic cell). Examples of cancers that are associated with solid tumor formation include breast cancer, uterine/cervical cancer, oesophageal cancer, pancreatic cancer, colon cancer, colorectal cancer, kidney cancer, ovarian cancer, prostate cancer, head and neck cancer, nonsmall cell lung cancer stomach cancer, tumors of mesenchymal origin (i.e; fibrosarcoma and rhabdomyosarcoma) thyroid cancer.

In the context of the present invention, tumors of the central and peripheral nervous system (i.e; including astrocytoma, neuroblastoma, glioma, glioblatoma) are not preferred type of tumors because Tau protein is also expressed by neural cells.

In previous study, it was shown that Tau is overexpressed in different human breast, gastric, prostate cancer cell lines and tissues (Gargini et al., 2019). In particular embodiment regarding the method of the present invention, the solid tumor is selected from the group consisting of breast cancer ((Rouzier et al., 2005; Matrone et al., 2010; Spicakova et al., 2010; Li et al., 2013), gastric cancer (Wang Q et al Pathol. Oncol. Res. (2013) 19:429-435), ovarian cancer (Smoter M. et al. Journal of Experimental & Clinical Cancer Research (2013), 32:25) and prostate cancer.

Tau protein level may be measured directly in a tumor sample or in blood sample obtained from the patient. Accordingly the biologic sample is tumor sample or blood sample; In a preferred embodiment the biologic sample is tumor sample.

### HDAC Inhibitors

As used herein, the term “response to a HDAC inhibitor treatment” refers to a clinically significant relief in the disease when treated with a HDAC inhibitor.

The term “histone deacetylase” or “HDAC”, as used herein, refers to an enzyme that removes acetyl groups from histones. There are currently 18 known HDACs, which are classified into four groups. Class I HDACs, includes HDAC1-3 and HDAC8. Class II HDACs include HDAC4-7 and HDAC9-10. Class III HDACs (also known as the sirtuins) include SIRT1-7. Class IV HDACs, which contains only HDAC11, has features of both Class I and II HDACs.

The term “histone deacetylase inhibitor” or “HDACi” as used herein, refers to a compound natural or synthetic that inhibits histone deacetylase activity. There exist different classes of HDACi in function of their selectivity for their substrates divided in classical HDACi, selective class I HDACi and selective class II HDACi.

A “classical HDACi” (also known as pan-HDACi) refers thus to a compound natural or not which has the capability to inhibit the histone deacetylase activity independently of the class of HDACs. Therefore a classical HDACi is a non selective HDACi. By “non selective” it is meant that said compound inhibits the activity of classical HDACs (i.e. class I, II and IV) with a similar efficiency independently of the class of HDAC. Examples of classical HDACi include, but are not limited to, Belinostat (PDX-101), Vorinostat (SAHA) and Panobinostat (LBH-589).

A “selective class I HDACi” is selective for class HDACs (i.e. HDAC 1-3 and 8) as compared with class II HDACs (i.e. HDAC4-7, 9 and 10). By “selective” it is meant that selective class I HDACi inhibits class I HDACs at least 5-fold, preferably 10-fold, more preferably 25-fold, still preferably 100-fold higher than class II HDACs. Selectivity of HDACi for class I or class II HDACs may be determined according to previously described method (Kahn et al. 2008). Examples of selective class I HDACi include, but are not limited to, valproic acid (VPA), Romidepsin (FK-228) and Entinostat (MS-275).

A “selective class II HDACi” is selective for class II HDACs (i.e. HDAC4-7, 9 and 10) as compared with class I HDACs (i.e. HDAC 1-3 and 8). By “selective” it is meant that selective class II HDACi inhibits class II HDACs at least 5-fold, preferably 10-fold, more preferably 25-fold, still preferably 100-fold higher than class I HDACs. Examples of selective class II HDACi include, but are not limited to, tubacin and MC-1568 (aryloxopropenyl)pyrrolyl hydroxamate).

HDAC inhibition relies mainly on a mechanism based on the inhibition of the HDAC enzymatic activity which can be determined by a variety of methods well known by the skilled person. Usually, these methods comprise assessing the lysine deacetylase activity of HDAC enzymes using colorimetric HDAC assays. Commercial kits for such techniques are available (see for example, Histone Deacetylase (HDAC) Activity Assay Kit (Fluorometric) purchased from Abcam or Sigma-Aldrich). These methods are ideal for the determination of IC50 values of known or suspected HDAC inhibitors.

Many HDAC inhibitors are known and, thus, can be synthesized by known methods from starting materials that are known, may be available commercially, or may be prepared by methods used to prepare corresponding compounds in the literature.

A preferred class of HDAC inhibitors are hydroxamic acid inhibitors which are disclosed e. g. in WO 97/35990, U.S. Pat. Nos. 5,369,108, 5,608,108, 5,700,811, WO 01/18171, WO 98/55449, WO 93/12075, WO 01/49290, WO 02/26696, WO 02/26703, JP 10182583, WO 99/12884, WO 01/38322, WO 01/70675, WO 02/46144, WO 02/22577 and WO 02/30879. All HDAC inhibitors disclosed in these publications are included herein by reference.

Other HDAC inhibitors which can be included within the compositions of the present invention are cyclic peptide inhibitors, and here it can be referred e. g. to U.S. Pat. Nos. 5,620,953, 5,922,837, WO 01/07042, WO 00/08048, WO 00/21979, WO 99/11659, WO 00/52033 and WO 02/0603. All HDAC inhibitors disclosed in these publications are included herein by reference.

Suitable HDAC inhibitors are also those which are based on a benzamide structure which are disclosed e. g. in Proc. Natl. Acad. Sci. USA (1999), 96: 4592-4597, but also in EP-A 847 992, U.S. Pat. No. 6,174,905, JP 11269140, JP 11335375, JP 11269146, EP 974 576, WO 01/38322, WO 01/70675 and WO 01/34131. All HDAC inhibitors, which are disclosed in these documents, are included herein by reference.

The HDAC inhibitors may be used under any pharmaceutically acceptable form, including without limitation, their free form and their pharmaceutically acceptable salts or solvates.

The term “pharmaceutically acceptable salts” refers to salts prepared from pharmaceutically acceptable, preferably non-toxic, bases or acids including mineral or organic acids or organic or inorganic bases. Such salts are also known as acid addition and base addition salts.”

Examples of pharmaceutically acceptable salts are discussed in Berge et al., 1977, “Pharmaceutically Acceptable Salts,” J. Pharm. Sci., Vol. 66, pp. 1-19.

The term “solvate” refers to a molecular complex comprising the drug substance and a stoichiometric or non-stoichiometric amount of one or more pharmaceutically acceptable solvent molecules (e.g., ethanol). The term “hydrate” refers to a solvate comprising the drug substance and a stoichiometric or non-stoichiometric amount of water.

More particularly, examples of suitable HDAC inhibitors according to the invention include, but are not limited to the compounds listed in Table 1 below:

TABLE 1
Examples of HDAC inhibitors useful
within the context of the invention

| Class | Agent |
|---|---|
| Hydroxamates | Belinostat (PDX-101) |
| Hydroxamates | Vorinostat (SAHA) |
| Hydroxamates | Panobinostat (LBH-589) |
| Hydroxamates | Givinostat (IFT2357) |
| Hydroxamates | Abexinostat (PCI-24781) |
| Hydroxamates | Trichostatin A (TSA) |
| Hydroxamates | Quisinostat (JNJ-26481585) |
| Hydroxamates | Pracinostat (SB939) |
| Hydroxamates | Resminostat (4SC-201) |
| Hydroxamates | Tefinostat (CHR-2845) |
| Hydroxamates | Dacinostat (LAQ-824) |
| Hydroxamates | CG200745 |
| Hydroxamates | CHR-3996 |
| Hydroxamates | Citarinostat (ACY-241) |
| Hydroxamates | Ricolinostat (ACY-1215) |
| Hydroxamates | MC-1568 |
| Benzamides | Mocetinostat (MGCD0103) |
| Benzamides | Entinostat (MS-275) |
| Benzamides | Tacedinaline (CI994) |
| Benzamides | Chidamide (CS055/HBI-8000) |
| Benzamides | BML-210 |
| Cyclic peptides | Romidepsin (FK-228, Depsipeptide) |
| Cyclic peptides | Trapoxin A |
| Cyclic peptides | Apicidine |
| Short-chain fatty acids (SCFA) | Sodium Phenylbutyrate |
| Short-chain fatty acids (SCFA) | Valproic acid (VPA) |
| Short-chain fatty acids (SCFA) | Sodium butyrate (S4125) |

In a preferred embodiment, the HDAC inhibitor is selected from the group consisting of belinostat (PXD-101), vorinostat (SAHA), entinostat (MS-275) panabinostat (LBH-589), mocetinostat (MGCD0103), chidamide (HBI-8000) romidepsin (FK-228) and Trichostatin A (TSA).

Belinostat (also known as PXD-101) has the chemical name (2E)-N-hydroxy-3-[3-(phenylsulfamoyl)phenyl]prop-2-enamide and has the following chemical formula:

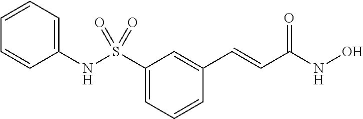 [SMILES: O=C(/C=C/c1cccc(S(=O)(=O)Nc2ccccc2)c1)NO]

Belinostat is currently commercially available for injection in the U.S. under the brand name Beleodaq® (Spectrum Pharmaceuticals). Typically, liquid formulations of belinostat comprise L-arginine, and are suitable for administration by injection, infusion, intravenous infusion, etc

Belinostat and pharmaceuticals compositions comprising thereof useful in the present combinations are described in the international patent applications N^{o} WO 2002/30879 and WO 2006/120456, the contents of both of which are incorporated herein in their entirety. In certain embodiments, belinostat is formulated with arginine (such as L-arginine).

Vorinostat (also known as suberoylanilide hydroxamic acid (SAHA)) has the chemical name N-hydroxy-N′-phenyloctanediamide and has the following chemical formula:

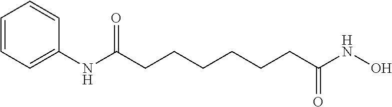 [SMILES: O=C(CCCCCCC(=O)Nc1ccccc1)NO]

Vorinostat is currently commercially available for oral administration in the U.S. under the brand name Zolinza® (Merck Sharp & Dohme Corp).

Panabinostat (also known LBH-589) has the chemical name 2-(E)-N-hydroxy-3-[4[[[2-(2-methyl-1H-indol-3-yl)ethyl]amino]methyl]phenyl]-2-propenamide and has the following chemical formula:

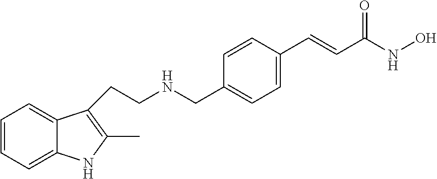 [SMILES: Cc1[nH]c2ccccc2c1CCNCc1ccc(/C=C/C(=O)NO)cc1]

Panabinostat lactate is currently commercially available for oral administration in the U.S. under the brand name Farydak® (Novartis).

Mocetinostat (also known as MGCD0103) has the chemical name N-(2-Aminophenyl)-4-[[(4-pyridin-3-ylpyrimidin-2-yl)amino]methyl]benzamide and has the following chemical formula:

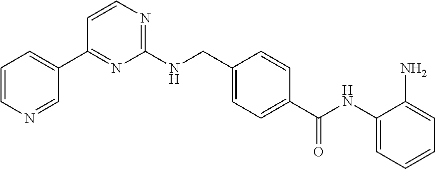 [SMILES: Nc1ccccc1NC(=O)c1ccc(CNc2nccc(-c3cccnc3)n2)cc1]

Mocetinostat has been used in clinical trials in various cancers such as Relapsed/Refractory Lymphoma among others.

Chidamide (also known as HBI-8000) has the chemical name N-(2-Amino-5-fluorophenyl)-4-[[[1-oxo-3-(3-pyridinyl)-2-propen-1-yl]amino]methyl]-benzamide and has the following chemical formula:

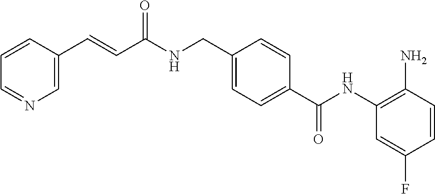 [SMILES: Nc1ccc(F)cc1NC(=O)c1ccc(CNC(=O)/C=C/c2cccnc2)cc1]

Chidamide is approved by the Chinese FDA for relapsed or refractory peripheral T-cell lymphoma (PTCL), under the brand name Epidaza® (Eisai).

Entinostat (also known as MS-275) has the chemical name N-(2-aminophenyl)-4-N-(pyridine-3-yl)methoxycarbonylamino-methyl]-benzamide and has the following chemical formula:

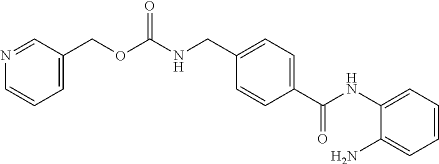 [SMILES: Nc1ccccc1NC(=O)c1ccc(CNC(=O)OCc2cccnc2)cc1]

Romidepsin is a natural product which was isolated from Chromobacterium violaceum by Fujisawa Pharmaceuticals. Romidepsin (also known as FK-228) is a bicyclic depsipeptide [1S,4S,7Z,10S,16E,21R)-7-ethylidene-4,21-bis(1methylethyl)-2-oxa-12,13-dithia-5,8,20,23-tetraazabicyclo[8.7.6]tricos-16ene-3,6,9,19,22-pentone] and has the following chemical formula:

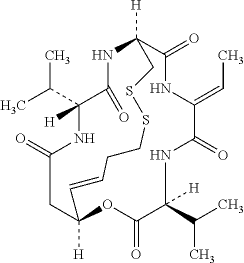 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

Trichostatin-A (TSA) (also known as TSA) has the chemical name of 2,4-Heptadienamide, 7-[4-(dimethylamino)phenyl]-N-hydroxy-4,6-dimethyl-7-oxo-, (2E,4E,6R)- and has the following chemical formula

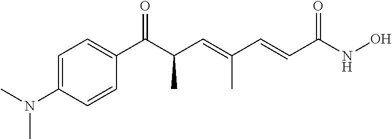 [SMILES: CC(/C=C/C(=O)NO)=C\[C@@H](C)C(=O)c1ccc(N(C)C)cc1]

TSA is an organic compound that serves as an antifungal antibiotic and selectively inhibits the class I and II mammalian histone deacetylase (HDAC) families of enzymes, but not class III HDACs (i.e., sirtuins). It is a member of a larger class of histone deacetylase inhibitors (HDIs or HDACIs) that have a broad spectrum of epigenetic activities. Thus, TSA has some potential as an anti-cancer drug (Drummond D C, et al. (2005) Annu Rev Pharmacol Toxicol. 45: 495-528.)

Vorinostat (see below) is structurally related to trichostatin A and used to treat cutaneous T cell lymphoma.

As used herein, the term “treating” or “treatment” means reversing, alleviating, inhibiting the progress of, or preventing the disorder or condition to which such term applies, or reversing, alleviating, inhibiting the progress of, or preventing one or more symptoms of the disorder or condition to which such term applies.

By a “therapeutically effective amount” is meant a sufficient amount to be effective, at a reasonable benefit/risk ratio applicable to any medical treatment. It will be understood, however, that the total daily usage will be decided by the attending physician within the scope of sound medical judgment. The specific therapeutically effective dose level for any particular patient in need thereof will depend upon a variety of factors including the age, body weight, general health, sex and diet of the patient, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

### Tau Protein and Inhibitor

As used herein, the term “Tau” denotes the Tau protein from mammals and especially from primates (and Tupaiidae). Human Tau is a neuronal microtubule-associated protein found predominantly in axons and functions to promote tubulin polymerization and stabilize microtubules. Six isoforms (isoform A, B, C, D, E, F, G, fetal-Tau) are found in the human brain, the longest isoform comprising 441 amino acids (isoform F, Uniprot P10636-8). Tau and its properties are also described by Reynolds, C. H. et al., J. Neurochem. 69 (1997) 191-198. Tau, in its hyperphosphorylated form, is the major component of paired helical filaments (PHF), the building block of neurofibrillary lesions in Alzheimer's disease (AD) brain. Tau can be phosphorylated at its serine or threonine residues by several different kinases including GSK3beta, cdk5, MARK and members of the MAP kinase family.

The protein sequence of human Tau protein, and its isoforms, may be found in Uniprot database with the following access numbers:
Tau isoform Fetal (352 Amino Acids) Uniprot P10636-2
Tau isoform B (381 AA) Uniprot P10636-4
Tau isoform D (383 AA) Uniprot P10636-6
Tau isoform C (410 AA) Uniprot P10636-5
Tau isoform E (412 AA) Uniprot P10636-7
Tau isoform F (441AA) Uniprot P10636-8
Tau isoform G (776 AA) Uniprot P10636-9

In human Tau protein is encoded by the MAPT (Microtubule associated protein tau) gene located on chromosome 17 (Gene ID: 4137). This gene has 18 transcripts (splice variants), 1 gene allele, 255 orthologues, 1 paralogue and is associated with 13 phenotypes.

Example of human MAPT transcripts which encoded Tau protein may be found in Ensembl database with the following access number
Transcript MAPT-201 (833 AA) Ensembl ID ENST00000262410 (Protein coding)
Transcript MAPT-202 (352 AA) Ensembl ID ENST00000334239 (Protein coding
Transcript MAPT-203 (736 AA) Ensembl ID ENST00000344290 (Protein coding)
Transcript MAPT-204 (441 AA) Ensembl ID ENST00000351559 (Protein coding)
Transcript MAPT-205 (776 AA) Ensembl ID ENST00000415613 (Protein coding)
Transcript MAPT-206 (412 AA) Ensembl ID: ENST00000420682 (Protein coding)
Transcript MAPT-207 (410 AA) Ensembl ID ENST00000431008 (Protein coding)
Transcript MAPT-208 (383 AA) Ensembl ID ENST00000446361 (Protein coding)
Transcript MAPT-209 (381 AA) Ensembl ID ENST00000535772 (Protein coding)
Transcript MAPT-212 (758 AA) Ensembl ID ENST00000571987 (Protein coding)
Transcript MAPT-214 (441 AA) Ensembl ID ENST00000574436 (Protein coding)
Transcript MAPT-217 (412 AA) Ensembl ID ENST00000680542 (Protein coding)
Transcript MAPT-218 (424 AA) Ensembl ID ENST00000680674 (Protein coding)

Of course variant sequences of the Tau may be used in the context of the present invention, those including but not limited to functional homologues, paralogues or orthologues of such sequences.

The term “Tau” should be understood broadly, it encompasses the native Tau, variants thereof having binding activity with core histone and fragments thereof having binding activity with core histone.

In particular the native Tau, variants and isoforms preferably contain at least three or four microtubule binding domains (named 3R and 4R respectively). All humanTau isoform and MAPT transcript above described contains at least three or four microtubule binding domains (see table 2).

In a particular embodiment the Tau protein used in the context of the present invention is transcript Variant (or Tau Isoform) selected from the list consisting of (1N4R) Transcript MAPT-206 (412 AA) (Protein coding Tau isoform E) and (2N4R) Transcript MAPT-214 (441 AA) (Protein coding Tau isoform F).

In particular, the binding to core histone of Tau occurs when H3 and H4 are devoid of any post-translational modifications or with acetylated H4 that increases the Tau's affinity

Typically a variant of Tau has at least 80%, preferably, at least 85%, more preferably at least 90% and even more preferably at least 95% identity with Tau.

Binding activity of Tau protein with core histone such as H4 can be measured for example as described in experimental section. Briefly, purified GST-Tau is incubated with biotin-labeled synthetic peptides corresponding to the N-terminal tail of histone H4 followed by incubation with M-280 streptavidin beads (Dynal). Peptide sequences were derived from human histone H4. Bound materials were resolved on SDS/PAGE and immunoblotted. Western-blot analysis can be carried out using primary antibodies directed against histone H3 (Millipore, 07-690), H4 (Santacruz, sc10810), H2A (Santacruz, sc8648), H2B (Active Motif, 39125), Tau C-ter (Galas et al., 2006) as described previously (Chauderlier et al., 2018).

As used herein, the term “Tau inhibitor” denotes a molecule or compound which can inhibit directly or indirectly the activity of the protein by limiting or impairing the interactions of the protein (ie with histone cores), or a molecule or compound which destabilizes the protein structure, or a molecule or compound which inhibits the transcription or the translation of Tau, or accelerates its degradation. The term “Tau inhibitor” also denotes an inhibitor of the expression of the gene coding for the protein.

A specific embodiment, “the Tau inhibitor, is a Tau inhibitor which directly binds to tau (protein or nucleic sequence (DNA or mRNA)) and neutralizes, blocks, inhibits, abrogates, reduces or interferes with the binding activity of Tau protein with core Histone.

Accordingly, in the context of the present invention, the Tau inhibitor (i) directly binds to Tau (protein or nucleic sequence (DNA or mRNA)) and (ii) inhibits binding activity of Tau protein with core histone. Examples of Tau inhibitors include but are not limited to any of the inhibitors described in “Jadhav et al. Acta Neuropathologica Communications (2019) 7:22”: all of which are herein incorporated by reference.

Typically, a tau inhibitor according to the invention includes but is not limited to:
A) Inhibitor of Tau activity selected from the list consisting Anti-Tau antibody and anti-Tau aptamers, tau peptide (vaccines)
B) PROTAC (“Proteolysis Targeting Chimera”) which simultaneously bind a tau protein and an E3-ubiquitin ligase.
C) Inhibitor of Tau gene expression selected from the list consisting of antisense, oligonucleotide, nuclease, siRNA, shRNA or ribozyme nucleic acid sequence.
   Antibody anti Tau

In some embodiment, the Tau inhibitor according to the invention is an antibody. Antibodies directed against Tau can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against TAU can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-TAU single chain antibodies. Compounds useful in practicing the present invention also include anti-TAU antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to Tau. Humanized anti-TAU antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Tau inhibitors such as anti Tau antibodies are well known in the art. Examples of patents disclosing anti Tau antibodies are. WO/2012/049570, WO/2014096321, WO/2015/004163; WO/2015200806, WO/2016/112078, WO/2018/152359, WO/2020/120644 (VHH anti Tau) WO/2020193520, WO/2021/010712, . . . .

In the context of the invention, it could be advantageous to use a nanobody directed against Tau in order to enter the cell. Thus in another embodiment, the antibody according to the invention is a single domain antibody directed against Tau. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH. The VHH according to the invention can readily be prepared by an ordinarily skilled artisan using routine experimentation. The VHH variants and modified form thereof may be produced under any known technique in the art such as in-vitro maturation. VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

Examples of patent disclosing VHH anti Tau antibodies is. WO/2020/120644

### Aptatmer

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

### Tau Polypeptide (Active Immunization)

In one embodiment, the compound according to the invention is a polypeptide. In a particular embodiment a Tau polypeptide may be used as vaccine composition in order to induce an anti Tau serum.

Accordingly, another example of Tau inhibitors according to the invention is a vaccine composition comprising an isolated peptide of Tau.

By “vaccine composition” it is herein intended a substance which is able to induce an immune response in an individual, and for example to induce the production of antibodies directed against the isolated tau polypeptide.

A vaccine is defined herein as a biological agent which is capable of providing a protective response in an animal to which the vaccine has been delivered and is incapable of causing severe disease. The vaccine stimulates antibody production or cellular immunity against the pathogen (or agent) causing the disease; administration of the vaccine thus results in immunity from the disease.

Active immunization with vaccine composition is long lasting because it induces immunological memory. Active vaccines are easy to administer (different routes) and the production is cost-effective. Immunization generates polyclonal response; antibodies can recognize multiple epitopes on the target protein with different affinity and avidity. On the other hand, the immune response depends on the host immune system, there is a variability in the antibody response across patients.

Like their passive immunotherapy counterparts, active vaccines targeting the mid-region, microtubule binding domain of Tau and C-terminus of Tau have been extensively investigated in preclinical studies (see table 3 of Jadhav et al. Acta Neuropathologica Communications (2019) 7:22).

There are two tau active vaccines that have been tested in human clinical trials, AADvac1 for Alzheimer's disease and non-fluent primary progressive aphasia (Axon Neuroscience SE), and ACI-35 vaccine for Alzheimer's disease (AC Immune SA, Janssen). Active vaccine AADvac1 consists of tau peptide (aa 294-305/4R) that was coupled to keyhole limpet haemocyanin (KLH) in order to stimulate production of specific antibodies. ACI-35 vaccine is a liposome-based vaccine consisting of a synthetic peptide to mimic the phospho-epitope of tau at residues pS396/pS404 anchored into a lipid bilayer.

### PROTAC Against Tau

The term “PROTACs” (“Proteolysis Targeting Chimera”) means bi-functional molecules which simultaneously bind a target protein and an E3-ubiquitin ligase. This causes the poly-ubiquitination of the target protein which is thus degraded into small peptides and amino acids by the proteasome complex. The PROTAC approach is therefore a chemical protein knock-down strategy.

It is therefore could be useful to provide bifunctional chimeric ligands capable of inducing targeted proteolysis of Tau according to the PROTAC strategy.

An example of PROTAC targeting Tau is describe Lu M. et al “Discovery of a Keap1-dependent peptide PROTAC to knockdown Tau by ubiquitination-proteasome degradation pathway European Journal of Medicinal Chemistry 146 (2018) 251e259. Briefly in this study authors identified Keap1, a substrate adaptor protein for ubiquitin E3 ligase involved in oxidative stress regulation, as a novel candidate for PROTACs that can be applied in the degradation of the nonenzymatic protein Tau. This peptide PROTAC by recruiting Keap1-Cul3 ubiquitin E3 ligase was developed and applied in the degradation of intracellular Tau. Peptide 1 showed strong in vitro binding with Keap1 and Tau.

### Inhibitor of Tau Gene Expression

In another embodiment, the Tau inhibitor according to the invention is an inhibitor of Tau gene expression.

Small inhibitory RNAs (siRNAs) can also function as inhibitors of Tau expression for use in the present invention. Tau gene expression can be reduced by contacting a subject or cell with a small double stranded RNA (dsRNA), or a vector or construct causing the production of a small double stranded RNA, such that Tau gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). As example, siRNAs directed against TAU are described in.

Ribozymes can also function as inhibitors of Tau gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of TAU mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides and ribozymes useful as inhibitors of TAU gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing TAU. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles are provided in Kriegler, 1990 and in Murry, 1991. Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter can also be, e.g., a viral promoter, such as CMV promoter or any synthetic promoters or natural promoters enabling a cell specific expression.

### Method for Predicting the Response to a HDAC Inhibitor Therapy

Another aspect of the invention relates to a method for predicting the response to a HDAC inhibitor treatment in a patient suffering from a cancer, comprising the step of determining in a biological sample obtained from said patient level of Tau expression, wherein the level of Tau protein expression is predictive of a response to a HDAC inhibitor treatment.

A high level of Tau protein is predictive of a non-response to a HDAC inhibitor treatment.

A low (or null) level of Tau protein is predictive of a response to a HDAC inhibitor treatment.

Tau protein level may be measured directly in a tumor sample or in blood sample obtained from the patient. Accordingly the biologic sample is tumor sample or blood sample. A recent study has shown that Tau plasma level correlate with brain metastase in metastatic breast cancer patient (see Darlix et al BMC cancer (2019) 19:110). Tau protein being circulating proteins, typical biological samples to be used in the method according to the invention are blood samples (e.g. whole blood sample, serum sample, or plasma sample). In a preferred embodiment said blood sample is a serum sample.

Typically, a normal and average Tau level in plasma is about 2.5 pg/ml (between 2.4 pg/ml and 2.6 pg/ml) (see Fossati S, et al. Alzheimers Dement (Amst). 2019; 11:483-492; Simrén J, et al., Alzheimers Dement. 2021; 17(7):1145-1156 and Zerr I, et al. Alzheimers Res Ther. 2021; 13(1):86.) when Tau serum levels is performed with immunoassay (digital ELISA) using Single Molecule Array (Simoa) technology.

In a preferred embodiment the biologic sample is tumor sample.

Once the biological sample from the patient is prepared, the level of Tau or a fragment thereof may be measured by any known method in the art.

For example, the concentration of Tau or a fragment thereof may be measured by using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, high performance liquid chromatography (HPLC), size exclusion chromatography, solid-phase affinity, Immunocytochemistry (ICC) etc.

In a particular embodiment, such methods comprise contacting the biological sample with a binding partner capable of selectively interacting with Tau or a fragment thereof present in the biological sample.

The binding partner may be generally an antibody that may be polyclonal or monoclonal, preferably monoclonal. Polyclonal antibodies directed against Tau or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against Tau can be prepared and isolated using any technique that provides for the production of antibody molecules by continuous cell lines in culture. Techniques for production and isolation include but are not limited to the hybridoma technique originally described by Kohler et al. Nature. 1975; 256(5517):495-7; the human B-cell hybridoma technique (Cote et al Proc Natl Acad Sci USA. 1983; 80(7):2026-30); and the EBV-hybridoma technique (Cole et al., 1985, In Monoclonal Antibodies and Cancer Therapy (Alan Liss, Inc.) pp. 77-96). Alternatively, techniques described for the production of single chain antibodies (see e.g. U.S. Pat. No. 4,946,778) can be adapted to produce anti-tau, single chain antibodies. Antibodies useful in practicing the present invention also include anti-Tau or fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to Tau. For example, phage display of antibodies may be used. In such a method, single-chain Fv (scFv) or Fab fragments are expressed on the surface of a suitable bacteriophage, e. g., M13. Briefly, spleen cells of a suitable host, e. g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk et al. (1990) Science, 249, 505-510. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena (1999) Clin Chem. 45(9):1628-50. Peptide aptamers consist of conformationally constrained antibody variable regions displayed by a platform protein, such as E. coli Thioredoxin A, that are selected from combinatorial libraries by two hybrid methods (Colas et al. (1996). Nature, 380, 548-50).

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labeled”, with regard to the antibody, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188.

The aforementioned assays generally involve the bounding of the binding partner (ie. Antibody or aptamer) in a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against Tau or a fragment thereof. A biological sample containing or suspected of containing Tau or a fragment thereof is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Different immunoassays, such as radioimmunoassay or ELISA have been described in the art.

Measuring the concentration of Tau (with or without immunoassay-based methods) may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is use. Once separated, Tau may be identified based on the known “separation profile” e. g., retention time, for that protein and measured using standard techniques. Alternatively, the separated proteins may be detected and measured by, for example, a mass spectrometer.

Commercial ELISA with an anti-human tau monoclonal antibody is available for example: Invitrogen (Tau (Total) Human ELISA Kit Catalog #KHB0041) or in Abcam (Human Tau ELISA Kit (ab273617).

The term “responder” patient, or group of patients, refers to a patient, or group of patients, who show a clinically significant relief in the disease when treated with a HDACi. Conversely, a “non responder patient” or group of patients, refers to a patient or group of patients, who do not show a clinically significant relief in the disease when treated with a HDACi.

Typically, a high or a low level of Tau is intended by comparison to a control reference value.

Said reference control values may be determined in regard to the level of Tau present in blood samples (or tissue sample) taken from one or more healthy subject or to the Tau distribution in a control population.

In one embodiment, the method according to the present invention comprises the step of comparing said level of Tau to a control reference value wherein a high level of Tau compared to said control reference value is predictive of a high risk of being a non-responder to a HDAC inhibitor treatment and a low level of Tau compared to said control reference value is predictive of a high risk of being responder to a HDAC inhibitor treatment.

In particular embodiment the level of Tau Expression detected in blood (or tumor sample) is null (or is not detected) using immunoassay-based methods.

The control reference value may depend on various parameters such as the method used to measure the level of Tau or the gender of the subject.

Typically and as illustrated in the Example section using a homemade tau Elisa KIT (see Elisa Measurement in Materiel and Methods section), in tumor MCF7 cell line which not respond to a HDAC inhibitor (Trichostatin A) the level of Tau protein in is egal to 20 ng/ml. Accordingly it could be expected that a level of Tau in a tumor sample lower than 5 ng/ml could be predictive of a low risk of being a non-responder to a HDAC inhibitor treatment.

Control reference values are easily determinable by the one skilled in the art, by using the same techniques as for determining the level of Tau in blood samples (or tumor sample) previously collected from the patient under testing.

A “control reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the βig-h3 levels (obtained according to the method of the invention) with a defined threshold value. In one embodiment of the present invention, the threshold value is derived from the Tau level (or ratio, or score) determined in a blood sample derived from one or more subjects who are responders to HDACi treatment. In one embodiment of the present invention, the threshold value may also be derived from tau level (or ratio, or score) determined in a blood sample (or tumor sample) derived from one or more subjects who are not affected with cancer. Furthermore, retrospective measurement of the Tau levels (or ratio, or scores) in properly banked historical subject samples may be used in establishing these threshold values.

“Risk” in the context of the present invention, relates to the probability that an event will occur over a specific time period, as in the conversion to being responder to a HDAC inhibitor treatment, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a cancer condition or to one at risk of being not responder to a HDAC inhibitor treatment. Risk evaluation can also comprise prediction of future clinical parameters, traditional laboratory risk factor values, or other indices of cancer, such as cellular population determination in peripheral tissues, in serum or other fluid, either in absolute or relative terms in reference to a previously measured population. The methods of the present invention may be used to make continuous or categorical measurements of the risk of conversion to being not responder to a HDAC inhibitor treatment, thus diagnosing and defining the risk spectrum of a category of subjects defined as being at risk for being not responder to a HDAC inhibitor treatment. In the categorical scenario, the invention can be used to discriminate between normal and other subject cohorts at higher risk for being not responder to a HDAC inhibitor treatment. In other embodiments, the present invention may be used so as to help to discriminate those being not responder to a HDAC inhibitor treatment from being responder to a HDAC inhibitor treatment.

The invention will be further illustrated by the following figures and examples. However, these examples and figures should not be interpreted in any way as limiting the scope of the present invention.

### FIGURES

FIG. 1: Tau inhibition increases MCF7 breast cancer cell line sensitivity to TSA. (A) Tau protein expression in MCF7, MCF7shctrl and shTau were quantify by Elisa. (B) Effect of 100 nM TSA (48 h) on cell death in the MCF7shctrl and MCF7shTau subclones. Cell death was determined by staining and flow cytometric analysis as described in the materials and methods. (C) Effect of 100 nM TSA (48 h) on apoptosis in the MCF7shctrl and MCF7shTau subclones. Apoptosis was determined by flow cytometric analysis of the PI-positive and Annexin-V-positive cells as described in the materials and methods. (D) Cell cycle distribution was determined by FACS analysis of combined propidium iodide and EdU staining in MCF7shctrl and MCF7shTau in the absence or presence 100 nM TSA, 48 h. Data are mean±SD **P<0.01***P<0.001. All results are representative of three independent experiments.

FIG. 2: Tau expression prevented TSA-dependent chromatin remodeling.

(A) Representative confocal sections of MCF7shctrl and shTau cells untreated or treated with 100 nM TSA for 24 h. DNA was revealed by DAPI and HP1α was immuno-localized. (B) Quantification of HP1α clusters per nuclei visualized as described previously and realized on three independent experiments. (C) GADD45a and (D) p21 (CDKN1a) expression in MCF7shctrl and shTau cells untreated or treated with 100 nM TSA for 24 h were analyzed by real-time PCR and normalized to RPLO. Results are expressed, relative to the basal activity set to 1, as the mean±SD of three independent assays. (E) Tau tethering prevents adjacent reporter gene activity induced by TSA. GAL4UAS responsive luciferase reporter Hela stable cell line was transfected with GAL4DBD (GAL4) or GAL4DBD-Tau4R and then, 24 h later, treated with TSA (600 nM) for 24 h. The luciferase activity was determined as described in in the Materials and Methods. (F) Ectopic Tau4R expression prevents adjacent reporter gene activity induced by TSA. The GAL4UAS responsive luciferase reporter Hela stable cell line was transfected with a plasmid encoding Tau4R for 24 h. Then, cells were treated with an increasing concentration of TSA, as indicated for 24 h. The luciferase activity was determined as described in the Materials and Methods. Data are mean±SD **P<0.01***P<0.001 vs. control.

FIG. 3: Histone H3 acetylation and Tau occupancy at different promoters

(A) Chromatin immunoprecipitations-qPCR analysis of H3 acetylation and Tau occupancy on the GAPDH promoter in MCF7shctrl cells in the indicated conditions. MCF7shctrl cells were subjected to cross-linking by 1% formaldehyde. Chromatin fragments were then immunoprecipitated using antibodies (Ab) against acetylated H3 (ac-H3) or Tau and analyzed by quantitative PCR for the presence of the GAPDH promoter. (B) Functional organization of the p21 promoter. (C) ChIP-qPCR analysis of H3 acetylation in MCF7shctrl or shTau cells or (D) Tau occupancy in MCF7shctrl cells on the p21 promoter. (E) ChIP-qPCR analysis of H3 acetylation or Tau occupancy on the stably transfected GAL4UAS responsive luciferase reporter transfected, or not, with Tau4R, then 24 h later treated with 100 nM TSA for 24 h. Cells were then subjected to cross-linking by 1% formaldehyde. Chromatin fragments were then immunoprecipitated using antibodies (Ab) against acetylated H3 or Tau and analyzed by quantitative PCR for the presence of the GAL4 UAS promoter. Quantification of enrichment is represented as fold-enrichment relative to IgG. Data are mean±SD **P<0.01***P<0.001

FIG. 4: Tau4R is associated with condensed chromatin.

(A) Analysis of the different histone H3 or (B) H4 post-translational modifications associated with Tau in the condensed chromatin fraction (600 mM). Eluted Tau4R complex obtained from the 600 mM fraction obtained were analyzed for H3 and H4 post-translational modifications using ELISA kits. Data are mean±SD **P<0.01 ***P<0.001. All results are representative of three independent experiments.

FIG. 5: Tau4R binds directly to histones.

(A) is a quantification from three independent experiments: representative Western blot of 1) Tau4R interacts with core histones 2) histone H3 and H4 and 3) Histone H4 tail peptides tested for Tau binding.). Data are mean±SD *P<0.05 **P<0.01 ***P<0.001.

FIG. 6: Frontotemporal lobar degeneration Tau mutations disrupt its interaction with histones.

(A) Single confocal sections of Hela cells transfected with GFP-HIP1β, with or without Tau4R or TauP301L and treated 24 h later with the TSA (300 nM) for 24 h. Tau C-terminus antibodies and GFP fluorescence were used to visualize total Tau protein and HP1β respectively. Representative images are shown. (B) Quantification of HP1β clusters per nuclei visualized as described previously and realized on three independent experiments. Data are mean±SD *P<0.05 ***P<0.001.

FIG. 7: Tau4R increases chromatin compaction.

(A) and (B) Densitometric analysis of the 1N fragments obtained in control and TSA-treated conditions were calculated from three independent experiments Data are mean±SD *P<0.05 **P<0.01.

### EXAMPLE

### Material & Methods

### Materials and Plasmids

pGEX vectors encoding Tau and Tau-deletion mutants and pGEX-CBP (aa 1202-1848) fused to GST tag were a kind gift from J. C. Lambert (INSERM U1167, Lille, France) and C. Smet-Nocca (UMR8576, Villeneuve d'Ascq, France) respectively. GFP-HPlbeta was a gift from Tom Misteli (Addgene plasmid #17651). pGL4.31[luc2P/GAL4 UAS/Hygro] was purchased from Promega. Short hairpin Tau and RNA Ctrl vectors were purchased from Santacruz. pcDNA3-Tau4R has been described elsewhere (Lippens et al., 2004; Chauderlier et al., 2018). GAL4-Tau4R was obtained by inserting an in-frame Tau1N4R (referred to as Tau4R) cDNA isoform into the pM GAL4 DNA-BD cloning vector (Clontech). Purified histones and recombinant histones were obtained from Epicypher and New England Biolabs respectively. Trichostatin A and BIX 01294 (Sigma) were reconstituted in dimethylsulfoxide and Tetracycline (Sigma) in ethanol.

### Cell Culture and Transfection

SH-SY5Y human neuroblastoma cells expressing Tau4R tagged with the streptavidin-binding peptide (SBP) (referred as SH-SY5Y-(SBP)Tau4R) (Chauderlier et al., 2018), SH-SY5Y-Tet-on-Tau4R, Hela cells, MCF7 and MDA-MB-231 were cultured in Dulbecco's Modified Eagle's Medium with 10% fetal bovine serum, 2 mM L-glutamine and 50 U/ml penicillin/streptomycin (Gibco) at 37° C. in 5% CO₂ humidified air. Transient and stable transfections experiments were performed using the lipofectamine 3000 reagent (Invitrogen). Transient luciferase assays were performed with the dual-luciferase assay system (Promega). For stable clones, luciferase activities were measured using the luciferase assay system (Promega) and normalized against protein concentration. To isolate stably transfected clones, Hela cells were transfected with the pGL4.31[luc2P/GAL4 UAS/Hygro](referred to as the GAL4UAS stable Hela cell line) and selected with hygromycin (200 μg/ml). Of 6 clones tested for reporter activity, one clone was chosen for further studies. MCF7 and MDA-MB-231 cells were transfected with short hairpin Tau or RNA Ctrl vectors and selected with puromycin (1 mg/ml). Clones were isolated and tested for Tau expression.

### In Vitro Chromatin Assembly

In vitro chromatin was obtained using the chromatin assembly kit (Active Motif) according to the manufacturer's guidelines. Assembled chromatin was then incubated with purified GST or GST-Tau and subjected to limited micrococcal nuclease digestion.

### Micrococcal Nuclease Assays

Nuclei, from SH-SY5Y-Tet-on-Tau4R, induced, or not, with 1 μg/ml tetracycline, 24 h before adding the indicated concentration of trichostatin A (24 h), were resuspended in TM2 (10 mM Tris at pH 7.4, 2 mM MgCl2 and protease inhibitor mixture) then CaCl2) was added to 1 mM and subjected to micrococcal nuclease digestion. The reaction was stopped with addition of 2 mM final EGTA and treated with RNase and then with Proteinase K. The purified DNA was electrophoresed through a 20 cm 1.5% agarose gel and visualized with ethidium bromide. Band intensities were quantified using the ImageJ Gel Analysis program.

### Salt Fractionation of Nucleosomes

Salt fractionation of nucleosomes was performed as described previously (Teves and Henikoff, 2012). Aliquots of each fraction were collected for western-blot analysis or DNA extraction. To analyze histone H3 and H4 post-translational modifications, the supernatants of each fraction was subjected to streptavidin pulldown of Tau fused in frame with the streptavidin binding peptide by incubation with 20 mL of M-280 streptavidin beads (Dynal) in TNE buffer (10 mM Tris-HCl, pH 7.5, 200 mM NaCl, 1 mM EDTA). Bound materials were eluted with biotin (Invitrogen) and analyzed with the EpiQuik™ Histone H3 or H4 Modification Multiplex Assay Kit (EpiGentek) according to the manufacturer's guidelines.

### RNA Preparation and Real-Time PCR

Total RNA was prepared using RNeasyMinikit (Qiagen). Reverse transcription (RT) was performed using random hexamers as recommended by the manufacturer (Applied Biosystems). cDNAs were analyzed by PCR amplification using the TaqMan PCR master mix (Applied Biosystems) and a mix of RPLO primers and probes. The different probes were purchased from Applied Biosystems (assay on demand kit). Reactions (40 cycles) and data analysis were carried out with an ABI Prism 7700 (PerkinElmer).

### Chromatin Immunoprecipitations

The ChIP protocol used was previously described (Lefebvre et al., 2006). Primers sequences for the pGL4.31[luc2P/GAL4 UAS/Hygro] promoter were: forward, 5′-TTCCGGAGTACTGTCCTCCG-3′ (SEQ ID N^{o}1), and reverse, 5′-GGTAGAATGGCGCTGGGCCC-3′ (SEQ ID N^{o}2); for p21 promoter: distal forward 5′-TGCTTCCCAGGAACATGCTTG-3′ (SEQ ID N^{o}3) and reverse 5′-CTGAAAACAGGCAGCCCAAGG-3′ (SEQ ID N^{o}4); proximal forward 5′-GCAGAGGAGAAAGAAGCCTG-3′ (SEQ ID N^{o}5) and reverse 5′-GCAGAGGAGAAAGAAGCCTG-3′ (SEQ ID N^{o}6); p21 (transcription start site) TSS forward: 5′-GCAGAGGAGAAAGAAGCCTG-3′ (SEQ ID N^{o}7) and reverse 5′-GCTCTCTCACCTCCTCTG3′ (SEQ ID N^{o}8); for GAPDH TSS: forward 5′-GGCTCCCACCTTTCTCATCC-3′ (SEQ ID N^{o}9) and reverse 5′-GGCCATCCACAGTCTTCTGG-3′ (SEQ ID N^{o}10). Antibodies used in the studies included the following: anti-acetylated H3 (Active motif, 39139) and anti-Tau1 (Millipore, MAB3420). All ChIP analyses were performed in at least two independent experiments.

### Peptide Binding Assay.

100 ng of purified GST-Tau was incubated with 2 g of biotin-labeled synthetic peptides corresponding to the N-terminal tail of histone H4 (Epicypher) in a buffer containing 50 mM Tris HCl at pH 7.5, 150 mM NaCl, 0.5 mM DTT, and 0.25% Nonidet P-40 for 4 h at 4° C., followed by incubation with 20 μl of M-280 streptavidin beads (Dynal). Peptide sequences were derived from human histone H4, amino acids 1-23 (SGRGKGGKGLGKGGAKRHRKVLR) (SEQ ID N^{o}11). Beads were washed, and bound material was eluted with 2× sample buffer. Bound materials were resolved on SDS/PAGE and immunoblotted as described below.

### Western-Blot Analysis

Western-blot analysis was carried out using primary antibodies directed against histone H3 (Millipore, 07-690), H4 (Santacruz, sc10810), H2A (Santacruz, sc8648), H2B (Active Motif, 39125), Tau C-ter (Galas et al., 2006) as described previously (Chauderlier et al., 2018).

### GST-Pulldown Assays y

GST-pulldown experiments were performed as described previously using 1 g of GST or GST-Tau proteins and the equivalent amount of indicated proteins (Chauderlier et al., 2018).

### Immunofluorescence

MCF7shctrl, MCF7shTau or transfected Hela cells with Tau4R and GFP-HP1α were fixed in 4% paraformaldehyde for 30 min at room temperature. Permeabilization was carried out in 0.2% Triton X-100 in phosphate-buffered saline for 10 min at room temperature. After 1 h saturation in 2% bovine serum albumin, immunostaining was performed using Tau antibody (recognizing the C-terminal domain of Tau). Tau staining was revealed with a goat anti-rabbit IgG antibody coupled to Alexa Fluor® 568 (Molecular Probes). Nuclear staining was performed by adding 1/2000 DAPI in phosphate-buffered saline for 10 min. Slides were then analyzed with a Zeiss LSM710 confocal laser scanning microscope (60× magnification). Images were collected in the Z direction at 0.80 μm intervals and quantifications were realized using the Image J plug-in.

### ELISA Measurements

Cellular levels of Tau protein were measured in serial dilution of cell lysates with a home-made tau ELISA kit. MCF7 shCtrl and shTau cells were lysed in 100 μL RIPA (150 mM NaCl, 1% NP40, 0.5% sodium deoxycholate, 0.1% SDS, 50 mM Tris-HCl, pH 8 and completed with protease inhibitors). Then, 96-well microtiter plates (Maxisorp F8; Nunc, Inc.) were coated overnight at 4° C. with 100 ng/well of our home-made antibody 9H12 (recognizing the 162-175 central region of tau) in a carbonate buffer (50 mM NaHCO₃, pH 9.6). After 5 washes with PBS containing 0.05% Tween (PBS-T), saturation was performed using 200 μL of PBS-T with 2% casein (PCT) for 1 hour at 37° C. After 5 washes with the PBS-T buffer, 50 μL of diluted cell lysates in PCT buffer were incubated for a further 1 hour at 37° C. The standard containing 1N4R recombinant tau were also incubated. Then, 50 μL of the detection antibodies (home-made TauE13N (7F5)) diluted at 1/1000 in PCT buffer were incubated overnight at room temperature. After 5 washes with PBS-T, followed a 1/32000 diluted anti-mouse IgG1 secondary antibody incubation. 100 μL of TMB solution (one pellet of tetramethyl benzidine (TMB) diluted in citrate/phosphate buffer supplemented by 1/5000 H₂O₂) was incubated. The reaction was stopped by addition of 50 μL of sulfuric acid. Optical density was measured with a spectrophotometer (Multiskan Ascent, Thermo Labsystem) at 450 nm.

### Cell Cycle Analysis, Cell Death and Apoptosis

Cell cycle, Cell death and Apoptosis were analyzed with the Click-It Plus EdU Alexa Fluor 647 kit (Thermofisher), Zombie NIR dye (Zombie NIR Fixable Viability Kit—Biolegend) and Annexin V-FITC Apoptosis Detection Kit II (Calbiochem) according to the manufacturer's guideline. Fluorescence was analyzed using a LSR FORTESSA X20 cytometer (Becton Dickinson).

### Statistical Analysis

Data are mean±SD. Statistical tests were carried out using GraphPad Prism software (GraphPAD Inc.). Statistical significance between groups was analyzed with Wilcoxon-Mann-Whitney test and Mann-Whitney tests. A p value less than 0.05 was considered significant.

### Results

### Tau Knock-Down Increases Breast Cancer Cell Line Sensitivity to the Pan-Histone Deacetylase-Inhibitor Trichostatin A.

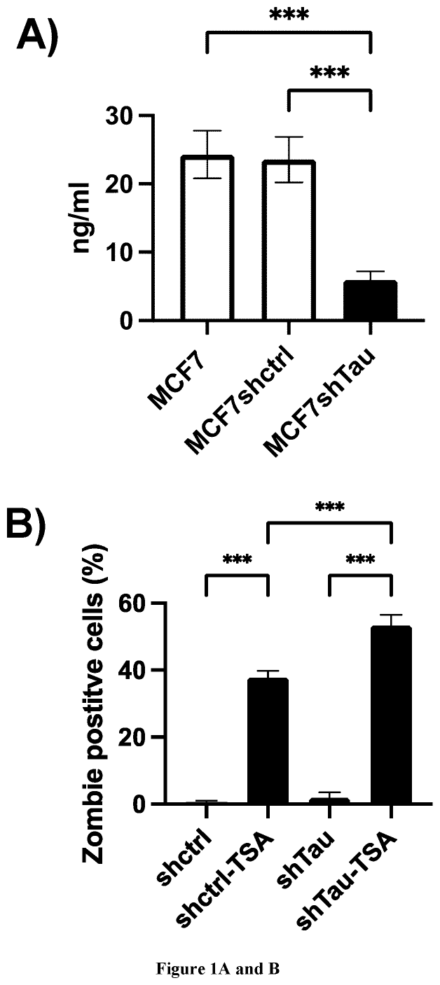

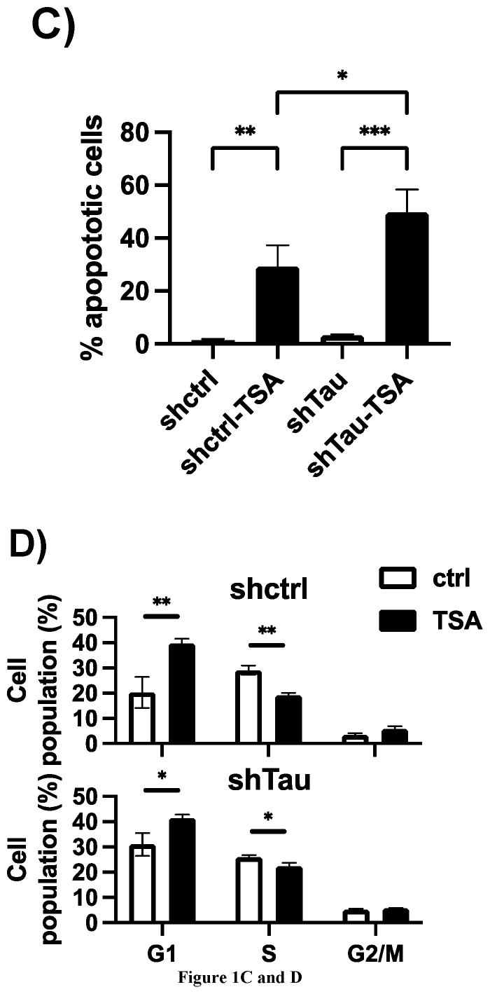

In this study we sought to investigated the role of Tau in controlling chromatin structure and functions. For this we took advantage of breast cancer cell lines that are known to express Tau. Indeed, Tau expression has been extensively characterized in breast cancer cell lines and tumors (Rouzier et al., 2005; Matrone et al., 2010; Spicakova et al., 2010; Li et al., 2013). We characterized the efficiency of Tau mRNA knock-down in several clones compared to the parental shctrl cell lines. Tau protein level decreased 75% in the selected MCF7shTau clones (FIG. 1A).

To dissect the role of Tau in controlling chromatin we sought to perturb chromatin structure by inhibiting histone deacetylation with the pan-histone deacetylase-inhibitor trichostatin A (TSA). In our preliminary experiments we found the lowest dose of TSA inducing cellular responses was 100 nM and we used this concentration in all the following experiments. First, we evaluated cell death, apoptosis and cell cycle progression by flow cytometric analyses in the two cell lines (as above we just illustrate our results with MCF7 cells in the main paper). We tested the effect of TSA in the presence or absence of short hairpin RNAs (Tau and Control). After 48 hours there was a strong increase in the number of dead cells from less 2% to >50% in the Tau-knockdown cells and a lesser increase to 38% in sh-ctrl cells (FIG. 1). In agreement, apoptosis was measured at 48% and 30% respectively (FIG. 1C).

To identify the mechanism of action of TSA in inducing cell death and apoptosis, we next performed cell cycle analyses (FIG. 1D). The results revealed that TSA treatment led to an accumulation of MCF7shctrl cells in G1 (39.6 vs. 20.2%) with a concomitant decrease in the S-phase (19.2 vs. 29.1%). However, no significant difference was observed in MCF7shTau cells after TSA treatment (41.4% for MCF7shTau vs. 39.6% for MCF7shctrl). This cell cycle analysis thus demonstrated that Tau expression did not impact on TSA-induced cell cycle arrest. Note that for all the above experiments we obtained similar results in MDA-MB-231 cells (data not shown).

Collectively, these results indicated that Tau inhibition increase TSA sensitivity towards apoptosis and cell death in different breast cancer cell lines independently of cell cycle arrest.

### Tau Prevents TSA-Dependent Chromatin Remodeling.

The above results suggested that Tau modulates the cellular responses to histone deacetylase inhibitors. As histone acetylation is known to affect the spreading heterochromatin-associated proteins, we next assessed the effect of trichostatin on the fate of the endogenous HP1α in shTau-knockdown MCF7 cells.

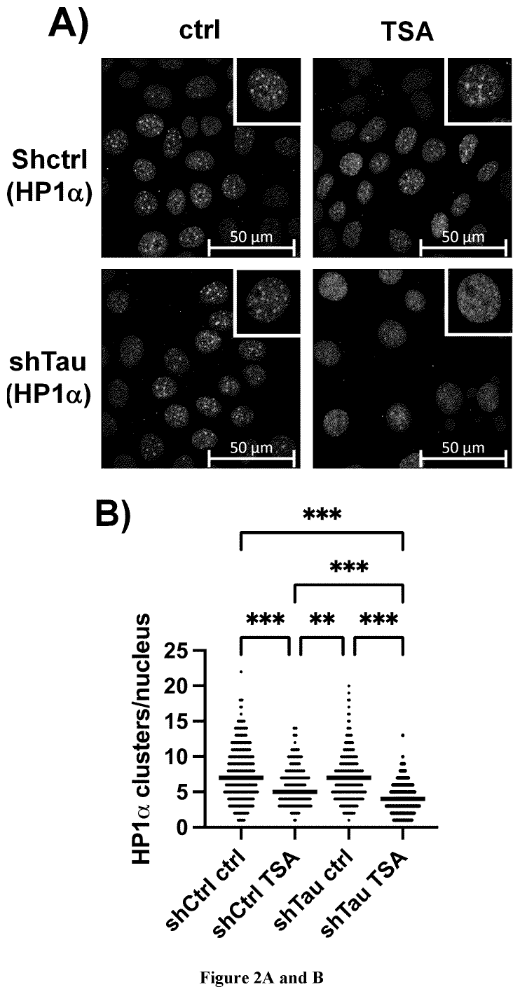

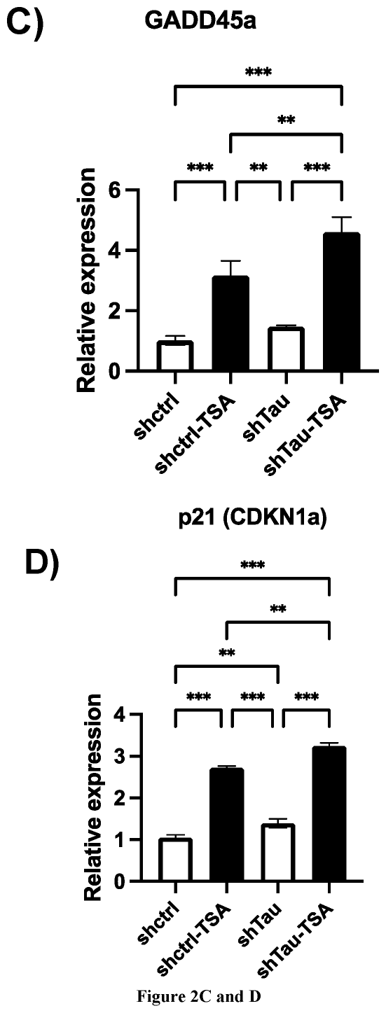

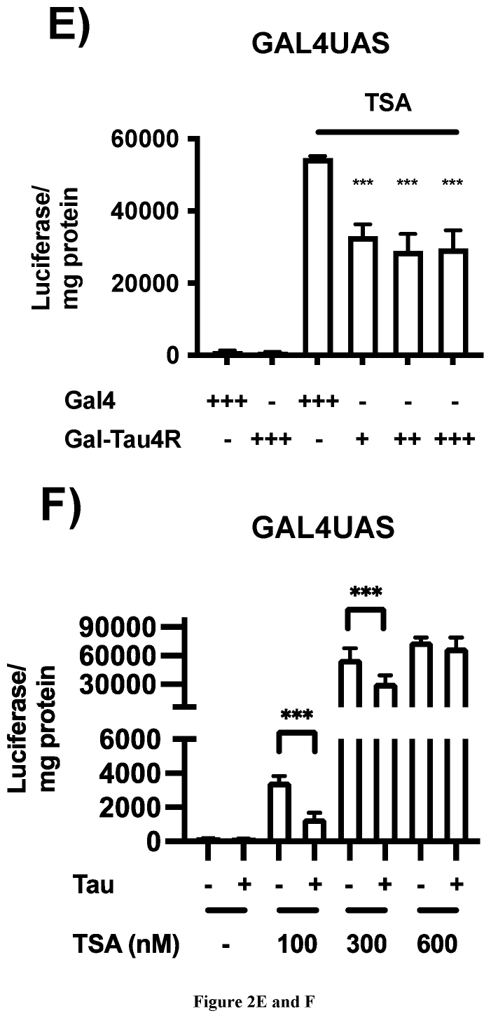

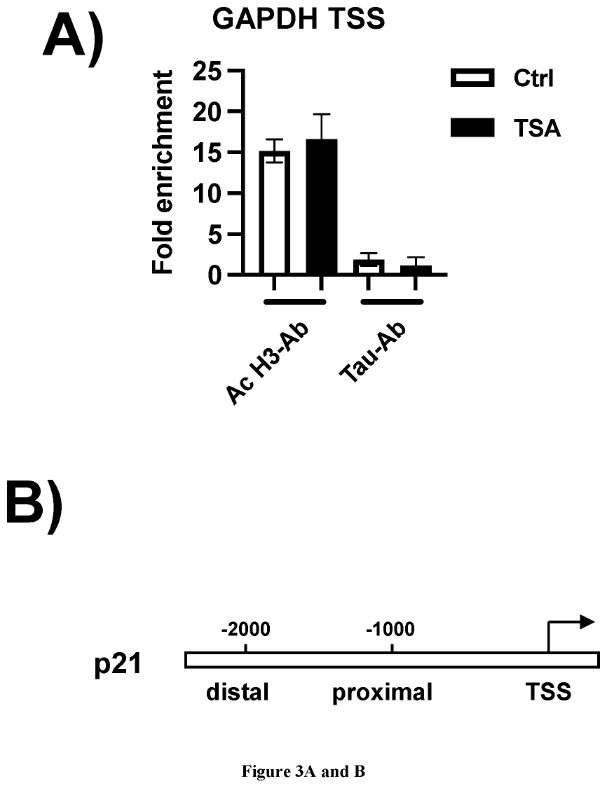

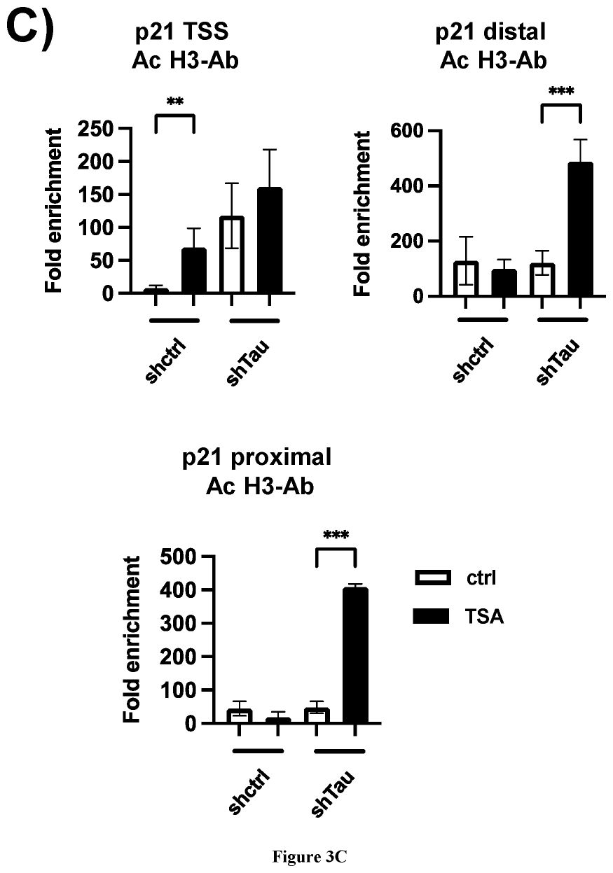

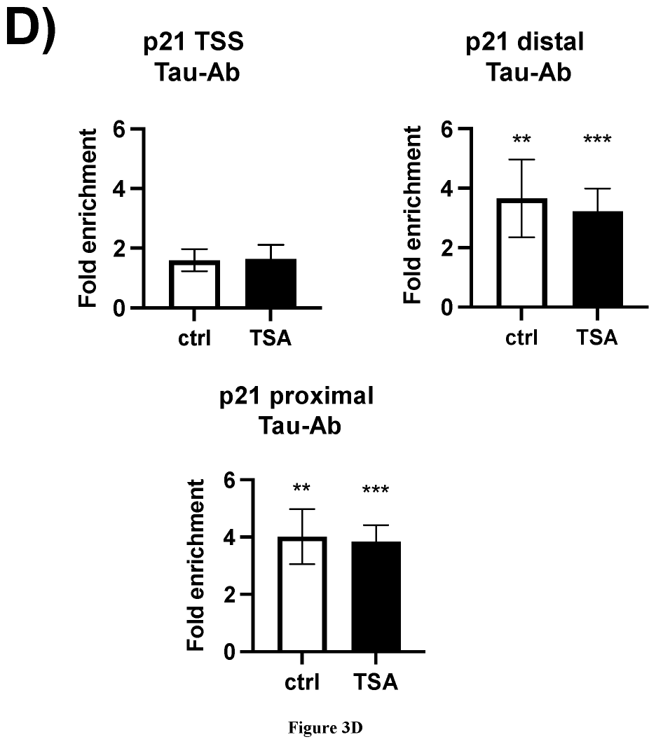

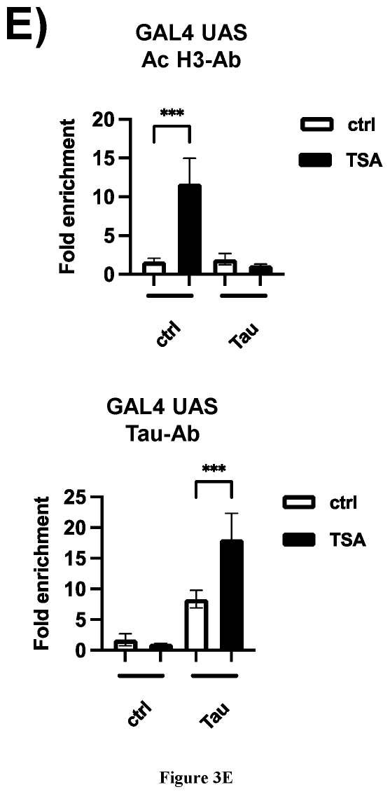

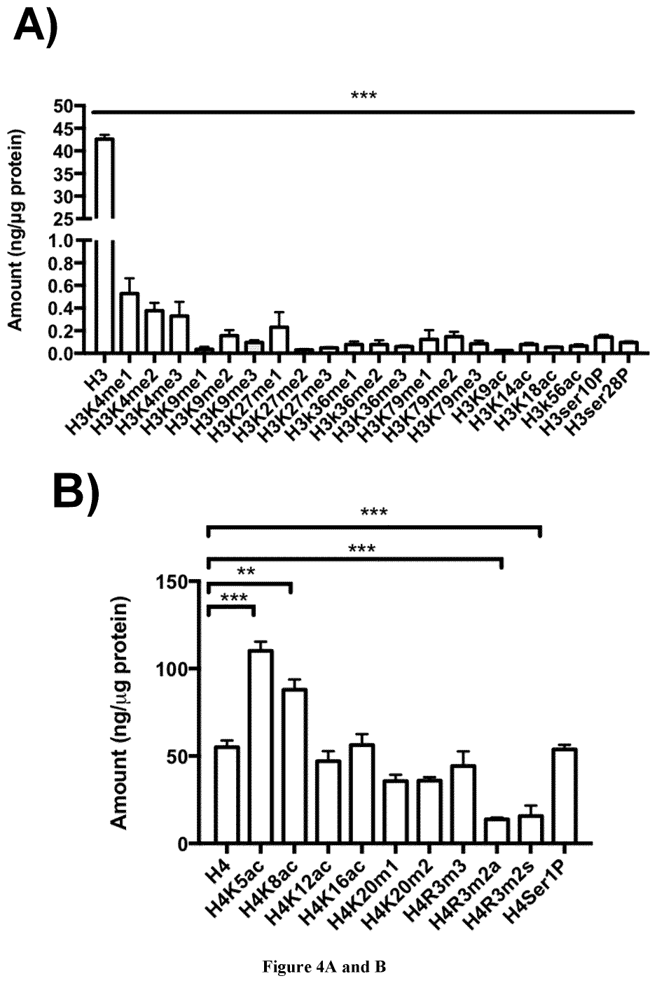

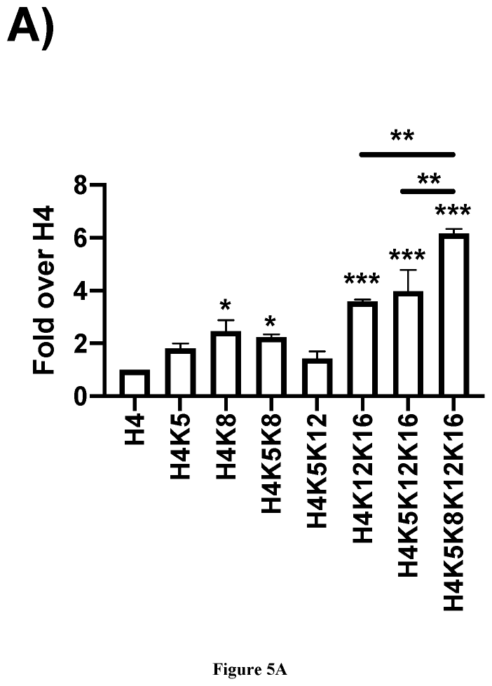

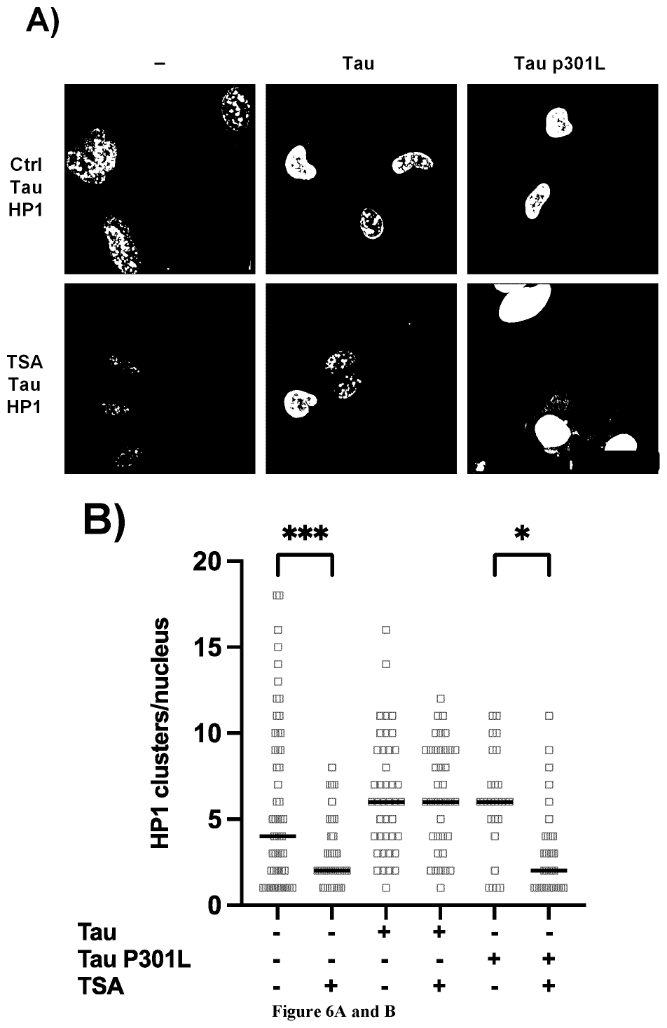

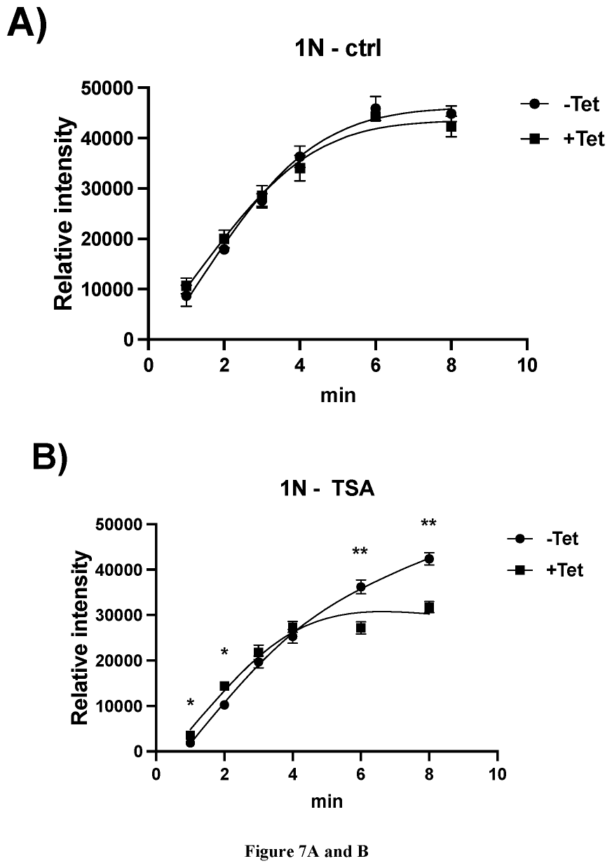

Without TSA, MCF7 cells had around eight HP1α clusters per nucleus in both sh-control and Tau-knockdown cells. TSA reduced this to five clusters and even further to just 3 HP1α clusters per nucleus where Tau was knocked down (FIG. 2A, B).

As well as disrupting pericentromeric heterochromatin, TSA affects histone acetylation at specific promoters and thereby influences chromatin structure and gene expression, of, for example, growth arrest DNA damage gene 45a (GADD45a) and p21 (cip/waf) (Richon et al., 2000; Hirose et al., 2003). As expected, these two genes were significantly upregulated by TSA in MCF7shctrl cells, as detected by RT-qPCR (3- and 2.5-fold induction respectively). Importantly this effect was exacerbated in cells with reduced Tau expression (FIGS. 2C and D). This indicates that Tau expression represses in part TSA-induced chromatin remodeling and gene expression.

To directly probe how Tau prevents TSA-induced chromatin remodeling and gene expression, we next used Hela cells as they lack endogenous Tau. First, we generated a stable GAL4UAS-responsive luciferase reporter Hela clone that we subsequently selected for trichostatin-induced luciferase activity. In our preliminary experiments we found the lowest dose of TSA inducing maximal luciferase activity was 600 nM and we used this concentration in the following experiments. In addition, by performing salt extraction on nuclei, we found only Tau4R isoforms were tightly bound to chromatin and could potentially impact directly on chromatin structure (data not shown). We therefore targeted Tau4R to UAS responsive elements by fusing Tau4R to the heterologous DNA-binding domain GAL4 and transfected this plasmid into Hela stable clone. Importantly we found the luciferase activity was significantly reduced (50%) in the presence of GAL-Tau4R (FIG. 2E). This demonstrates that the presence of Tau on the promoter reduced TSA-induced gene expression. We next wondered if this effect was due to the tethering of Tau4R to the promoter or whether this was independent of Tau4R fusion to GAL4. To this end, the wild-type Tau4R (i.e. without the GAL4 fusion) was transfected into a Hela clone and treated with various concentrations of TSA, up to 600 nM. Tau4R overexpression reduced the luciferase activity at 100 and 300 nM by around 50% decreased, whereas at 600 nM TSA there was no reduction (FIG. 2F). The fact that the presence of Tau4R decreased the TSA-inducibility of the GAL4UAS responsive luciferase reporter gene suggests that Tau, in particular Tau4R isoforms, modulated the effects of histone acetylation on gene expression.

### Tau4R Regulates Histone Deacetylase Inhibitor Induced Genes Through Direct Binding to Chromatin.

The above results suggest that Tau4R binds directly to chromatin to regulate gene expression. To test this hypothesis, we next performed chromatin immunoprecipitations assays. Antibodies specific for acetylated H3 (ac-H3) and Tau were used to immunoprecipitate formaldehyde-cross-linked sonicated chromatin from short hairpin Tau knockdowns and control cells treated, or not, with 100 nM TSA. Quantitative PCR analysis of input or immunoprecipitated DNA using acetylated H3 and Tau antibodies was carried out to detect different promoter regions. As a control of non-TSA-inducible gene, we first used primers encompassing the transcription start site of the GAPDH promoter. No change in the level of acetylated H3, nor Tau binding, were detected after TSA treatment (FIG. 3A). We next examined the extent of acetylated H3 and Tau occupancy within the p21 promoter associated chromatin that is illustrated schematically in FIG. 3B. As shown in FIG. 3C, TSA induced a strong acetylation in shctrl cells (70- vs. 7-fold enrichment compared to IgG) while a high level of acetylated H3 was detected in shTau cells, irrespective of TSA treatment (≈160-fold enrichment). Moreover, Tau was barely detectable at the p21 transcription start site region in MCF7shctrl cells, expressing Tau proteins (less than 1.4-fold enrichment compared to IgG ctrl) (FIG. 3D). qPCR analyses were then performed to detect the p21 proximal and distal regions. A significant increase in acetylated H3 was observed in MCF7shTau cells (≈10-fold enrichment compared to non-treated cells) but not in MCF7shctrl cells (FIG. 3C). In addition, Tau was detected at the level of these regions in MCF7shctrl cells (≈4-fold enrichment) in absence or presence of TSA treatment (FIG. 3D). These results are consistent with our hypothesis that Tau4R modulated the TSA induced-histone acetylation by binding to chromatin.

We next performed chromatin immunoprecipitations in the GAL4UAS responsive luciferase reporter Hela stable cell line for which we observed similar gene response after exogenous Tau4R expression and TSA treatment (see FIG. 2 above). We performed qPCR as described previously to detect a fragment of the integrated promoter, encompassing the GAL4UAS responsive elements and the transcription start site (FIG. 3E). A total of 100 nM TSA caused a ten-fold increase of ac-H3 in the control HeLa cells that was abrogated in the presence of transfected Tau4R. As expected, Tau4R was specifically enriched in the basal and TSA-treated conditions (7-fold and 16-fold respectively).

Taken together, these data demonstrated that Tau bound various DNA regions and inhibited histone H3 TSA-induced acetylation.

Tau4R Associates with Condensed Chromatin.

It has been demonstrated that chromatin fraction extracted at low salt concentration after micrococcal nuclease digestion solubilized active chromatin, the high-salt fraction was enriched in condensed/inactive chromatin while the remaining pellet contained transcriptionally active chromatin (Henikoff et al., 2009). We therefore sought insights into Tau4R's distribution on chromatin. To do this we digested and fractionated stable SH-SY5Y cells overexpressing Tau4R fused in frame with the streptavidin binding peptide (SH-SY5Y-(SBP)Tau4R), allowing the elution of Tau4R-associated proteins. As expected, DNA analyses showed that low salt fractions (80 and 150 mM) were mostly enriched in mononucleosomes, representing the active chromatin, while the high-salt fraction (600 mM) contained exclusively polynucleosomes, i.e. condensed chromatin (data not shown). In addition, western-blot analysis demonstrated that Tau was detected in all the fractions tested, as was histone H3 (data not shown). We next wondered if Tau4R might be specifically interacting with nucleosomes in the different chromatin states. For this, we performed streptavidin pulldown of the Tau fusion and tested the presence for Tau itself and that of nucleosomes (by the presence of H3) by western-blot analysis. Immunoprecipitated H3 was almost exclusively in the high-salt fraction while Tau was more evenly present in all fractions. These results suggested that Tau was preferentially associated with condensed chromatin. Several hypotheses could explain this. Previous reports demonstrated that Tau can bind DNA in vitro and could be associated with GAGA responsive elements (GAGA-RE) (Benhelli-Mokrani et al., 2018). However, we found no preferential binding for GAGA-RE compared to control sequence using microscale thermophoresis (data not shown). We therefore next considered the possibility that Tau could be associated with condensed chromatin because of post-translational modifications of histones, highly specific to the different chromatin compartments. After streptavidin pulldown of SBP-Tau4R, bound materials were eluted with biotin and tested for the presence of post-translational modifications of H3 and H4 with commercial ELISA kits. Non-modified H3 was present but none of the tested post-translational modifications were detected (FIG. 4A). For H4 there was almost no signal for H4R3me3a and m2s. However, all the other tested modifications were strikingly present in the Tau immunoprecipitate (FIG. 4B). Note that unmodified H4 signal was weaker than certain H4 post-translational modifications, suggesting that they might interfere with total H4-ab binding. However, normalization against total H3 did not modify the obtained results.

Taken together, these data place Tau4R in condensed chromatin regions containing acetylated H4 and unmodified H3.

### Tau4R/Chromatin Interaction is Mediated Through Histones.

Naturally our next hypothesis then was that tau could be associated with chromatin through direct interaction with histones. To address this possibility, we first realized GST-pulldown analysis using unmodified core histones. Tau4R but not GST alone bound specifically to the histone core as revealed by H3, H4, H2A and H2B antibodies. Using recombinant histones, we also showed that this interaction was mediated through H3 and/or H4 and not H2A and H2B (data not shown). We next tested the effect of different combination of H4 acetylation sites, not present in the H4 ELISA kit, on Tau4R interaction using synthetic H4 tail peptides. As seen in FIG. 5A, Tau bound to unacetylated H4 and H4K5acK12ac. In contrast to H4K5ac, acetylation at K8 (H4K8) slightly increased Tau binding (2-fold over unacetylated H4). Tau binding was not further increased by acetylation at position 5 (H4K5acK8ac). We observed a 4-fold increase in Tau binding for diacetylated H4 at position 12 and 16. Again, no change was observed when adding an acetyl group at position 5. However, a 6-fold increase was observed when using a tetra-acetylated H4. These results indicated that Tau binds H4 when unacetylated, but displays more affinity for tetra-acetylated H4.

### The Frontotemporal Lobar Degeneration Tau Mutation Abolished Tau/Histone Interaction.

Pericentromeric heterochromatin disruption was observed in neurons from frontotemporal lobar degeneration (Tau P301L/S) pathological models (Frost et al., 2014; Mansuroglu et al., 2016). These observations further suggest that P301L/S mutations, could nevertheless abolish Tau/histone interaction. To this end, we first performed GST-pulldown analysis using purified core histones. As shown previously, Tau interacted specifically with core histones as detected by H3, H4, H2A and H2B antibodies (data not shown). However, this interaction was greatly decreased by the TauP301L mutation. Based on these observations, we hypothesized that TauP301L mutant would not prevent HP1 spreading induced by TSA treatment. To test this, we next followed the fate of transfected GFP-HIP1β, in the absence or presence of transfected Tau4R or TauP301L mutant, following TSA treatment in Hela cells, which as we have mentioned are devoid of endogenous Tau expression. As shown above, we observed a decrease in the number of HP1 clusters in control cells following TSA treatment. This decrease was prevented when wild-type Tau was expressed, but not by the Tau P301L mutant (FIGS. 6A and B).

Taken together, these data demonstrated a direct relationship between Tau/histone interaction and TSA-induced chromatin remodeling.

### Tau4R Increases Chromatin Compaction.

A possible mechanism of the inhibitory effect on H3 acetylation is that Tau4R binding to histone blocks acetylation by steric hindrance. To test this hypothesis, we next performed in vitro acetyltransferase assays using core histone, unmodified or H4 tetra-acetylated nucleosomes. However, no change in H3 or H4 acetylation level was observed in the absence or presence of recombinant Tau4R (data not shown).

Another intriguing possible mechanism is that Tau4R binding to histones would induce chromatin compaction that prevent H3 acetylation. To address this question, we again used our micrococcal nuclease assay as the outcome is highly dependent on chromatin compaction. It has been shown that the cleavage pattern as well as the rate of conversion of chromatin into smaller nucleosome fragments reflected the accessibility of linker DNA to the enzyme and overall compactness of chromatin (Bryant, 2012).

To probe the effect of Tau on chromatin structure, this time we performed the nuclease assay using different time points. To control Tau4R expression, we therefore used SH-SY5Y Tet-on Tau4R cells. We induced Tau4R expression with tetracycline and then added 300 nM TSA, 24 h later. Then, we subjected an equivalent number of isolated nuclei to micrococcal nuclease digestion for different times (1, 2, 3, 4, 6 and 8 min) and compared the abundance of nucleosomal DNA corresponding to mono-, di-, tri- and tetranucleosomes. In the absence of TSA, chromatin from control and Tau4R expressing cells cleaved in a similar pattern at a similar rate (FIG. 7A and data not shown). Importantly however, TSA induced significant differences. There were less oligo-nucleosomes in Tau4R expressing cells, demonstrating that the chromatin was less accessible to micrococcal nuclease, meaning more compact, when Tau4R was present (FIG. 7B and data not shown).

To confirm our results were not due to experimental artefacts in our cell lines, we next used in vitro reconstituted chromatin incubated with purified recombinant GST or GST-Tau4R proteins in a micrococcal nuclease assay. Digestion with GST alone for 2 min produced a ladder of DNA fragments, mostly corresponding to mono-, di-, tri- and tetra-nucleosomes whereas the longer incubation (4 min) gave rise mostly to mono and di-nucleosomes (data not shown). Strikingly, when in vitro reconstituted chromatin was pre-incubated with purified GST-Tau4R, no ladder was observed after 2- or 4-min incubation with micrococcal nuclease, showing that chromatin in this condition was less accessible. These observations are consistent with a role of Tau4R in maintaining a compacted chromatin structure that prevents histone acetylation.

### DISCUSSION

An extensive body of literature suggested a possible role of Tau in chromatin functions and/or organization in neuronal, non-neuronal cells and cancer cells (Frost et al., 2014; Bukar Maina et al., 2016; Bou Samra et al., 2017; Klein et al., 2019). However a clear mechanism has not been demonstrated. Here we show that Tau preferentially binds to the condensed nuclease-resistant chromatin fraction devoid of any silent specific histone methylation. In addition, we demonstrated that Tau binding to histones is direct, involving unmodified histone H3 and H4 or tetra-acetylated H4 that increased interaction. As consequences, Tau 4R stabilizes condensed chromatin and heterochromatin.

Our results point out that Tau expression itself does not induce broad changes in chromatin organization/structure (e.g. chromatin accessibility, pericentromeric heterochromatin integrity and histone acetylation) and the effect of Tau was seen only when deacetylation was inhibited. These data suggest a more general role for Tau in preventing chromatin remodeling. In this regard, we found the same results using BIX 01294, a specific inhibitor of G9a histone methyltransferase catalyzing the di-methylated state of H3 at lysine 9 and known to disrupt pericentromeric heterochromatin (Kubicek et al., 2007) (data not shown). From these observations, we can also exclude a possible specific effect of trichostatin A on chromatin remodeling. In a drosophila model of tauopathy, heterochromatin disruption was hypothesized to be a consequence of oxidative stress/DNA damage and this effect could not be prevented by aggregated Tau proteins. Interestingly, there is now evidence suggesting that oxidative stress globally influences chromatin structure and enzymatic post-translational modifications of histones (Kreuz and Fischle, 2016).

In drosophila, global changes in gene expression have been also observed, indicating chromatin remodeling is not limited to pericentromeric heterochromatin structure. These observations were recapitulated in part in our cellular models. Trichostatin-induced gene expression was higher in cellular MCF7 models depleted of endogenous Tau for GADD45a and p21, two well-characterized histone deacetylase-inhibitor-inducible genes. In addition, luciferase reporter activation was observed after trichostatin A treatment and decreased in the presence of GAL4-tethered or wild-type Tau4R proteins. Note also that we did not observe an effect of GAL-Tau4R when the GAL4UAS responsive luciferase reporter Hela stable cell line displayed a high basal luciferase, representing state associated with active chromatin (data not shown). This observation suggests that Tau4R was unable to repress gene expression on active genes but rather maintains the condensed chromatin state.

Our results clearly demonstrated that Tau was mostly associated with condensed chromatin, where associated histones were devoid of any H3 post-translational modifications and could present in particular H4 acetylation marks. Our in vitro experiments demonstrate that Tau4R binds to unmodified core histones, but the affinity increased with H4 acetylation. Using genome-wide chromatin immunoprecipitation followed by microarray hybridization assays in primary neuronal culture, Benhelli-mokrani et al. suggested that an AG-rich GAGA-like DNA motif could play a role in Tau genomic localization (Benhelli-Mokrani et al., 2018). However, we found no correlation between the presence of GAGA sequences and Tau binding (supplementary Table 1). Our observation is more consistent with a previous report demonstrating that Tau DNA binding is sequence independent, involving just the DNA backbone (Qi et al., 2015). Sequence-independent DNA binding cannot explain the observed specific genomic distribution observed by Benhelli-Mokrani. The interaction with histones and nucleosome core particles we reveals likely confer additional specificity for Tau binding to chromatin.

The exact modality of Tau4R binding to histones H3 and H4 remains to be identified, since no structural similarities with other histone associated proteins were found. Since we did not observe any chromatin association with the Tau3R isoforms, we suspect an essential role of the second microtubule domain encoded by the exon 10 in the recognition process. This observation is reinforced by the loss of Tau/histone interaction observed with the P301L mutant. The mutation occurs within exon 10 and only affects Tau4R isoform, exon 10 being splice out of 3R isoforms (Sergeant et al., 2005). Note that the TauP301L/S mutations do not interfere with Tau nuclear localization (Siano et al., 2019).

Although Tau contains an intrinsic acetyltransferase activity, it did not appear to contribute directly to this specific acetylation pattern as demonstrated by our in vitro acetyltransferase assays (Cohen et al., 2013). It was quite surprising to find H4K16 acetylation in these nuclease resistant fractions, as this is a histone post-translational modification known to contribute directly to chromatin decompaction (Shogren-Knaak et al., 2006; Yu et al., 2011). However, there is substantial evidence that H4K16 acetylation does not alter higher chromatin compaction in vivo but rather it disrupts local chromatin structure (Taylor et al., 2013; Mishra et al., 2016). In addition, H4 acetylation has been detected in some heterochromatin compartments (Turner et al., 1992; Johnson et al., 1998). Most strikingly, our results show that Tau associated histones were devoid of the H3K9me2/3 hallmark of heterochromatin. This further suggests an indirect role for Tau4R in maintaining heterochromatin integrity.

Our results presented here indicate that Tau binding specifically inhibits H3 acetylation in cellulo, as shown for the p21 promoter (distal and proximal) in MCF7 cells and for the GAL4UAS responsive luciferase reporter in Hela cells. Tau was not detected at the level of p21 transcription start site, a region shown to be enriched in H3K4me3, which supports our idea that post-translational modification of H3 prevents Tau binding (Itahana et al., 2016). However, this effect was not seen not for nucleosomes or histones in vitro. On the balance of the data presented here we feel that the chromatin's condensation state prevents chromatin remodeling complexes accessing histones. Although our experiments demonstrate that histone acetylation is inhibited by Tau, other post-translational modifications of H3 are also likely inhibited as there are not seen in our streptavidin pull-down assay.

As a consequence of a cellular role of Tau in chromatin organization and/or function, we showed that Tau4R depletion in the luminal MCF7 or triple-negative MDA-MB-231 breast cancer cell lines increased TSA-induced cell death and apoptosis. Previous studies demonstrated that histone deacetylase-inhibitors led in particular to the up-regulation of pro-apoptotic genes (Li and Seto, 2016). We found that GADD45a, a gene that may be involved in apoptosis, was more expressed in MCF7shTau cells after TSA treatment (Takekawa and Saito, 1998; Zhang et al., 2001). Although p21 is known to be induced by inhibiting histone deacetylases, it is not clearly known how it controls the resulting apoptosis, although p21 can induce G1 arrest. Depending on the cellular model, G1 arrest could be protective or necessary for TSA-induced apoptosis (Peart et al., 2005) (Newbold et al., 2014). In this regard, we found no correlation between p21 expression, G1 arrest and the extent of apoptosis in MCF7 and MDA-MB-231 short hairpin cells.

In conclusion, this is the first study describing a role and underlying mechanism of Tau protein in chromatin structure and opens new avenues to further understand Tau biology in neuronal and cancer cells.

TABLE 2
Differents Human Tau isoforms

| Human Isoform Tau | | |
|---|---|---|
| (Binding domain) | Ensembl ref. ID | UniProt ref. ID |
| fetal Tau (0N3R) | ENST00000334239, | [P10636-2] |
| | ENSP00000334886, | |
| | ENSG00000186868 | |
| Tau F-2N4R | ENST00000351559, | [P10636-8] |
| | ENSP00000303214, | |
| | ENSG00000186868 | |
| Tau G | ENST00000415613, | [P10636-9] |
| | ENSP00000410838, | |
| | ENSG00000186868 | |
| Tau E 1N4R | ENST00000420682, | [P10636-7] |
| | ENSP00000413056, | |
| | ENSG00000186868 | |
| Tau C-2N3R | ENST00000431008, | [P10636-5] |
| | ENSP00000389250, | |
| | ENSG00000186868 | |
| Tau D-0N4R | ENST00000446361, | [P10636-6] |
| | ENSP00000408975, | |
| | ENSG00000186868 | |
| Tau B-1N3R | ENST00000535772, | [P10636-4] |
| | ENSP00000443028, | |
| | ENSG00000186868 | |
| PNS-Tau | ENST00000571987, | [P10636-1] |
| | ENSP00000458742, | |
| | ENSG00000186868 | |
| Tau F-2N4R | ENST00000574436, | [P10636-8] |
| | ENSP00000460965, | |
| | ENSG00000186868 | |
| Tau E-1N4R | ENST00000612872, | [P10636-7] |
| | ENSP00000478602, | |
| | ENSG00000277956 | |
| Tau E-1N4R | ENST00000613360, | [P10636-7] |
| | ENSP00000483784, | |
| | ENSG00000276155 | |
| Tau F-2N4R | ENST00000620070, | [P10636-8] |
| | ENSP00000484491, | |
| | ENSG00000277956 | |
| Tau C-2N3R | ENST00000620818, | [P10636-5] |
| | ENSP00000484321, | |
| | ENSG00000277956 | |
| Tau C-2N3R | ENST00000620981, | [P10636-5] |
| | ENSP00000481769, | |
| | ENSG00000276155 | |
| Tau F-2N4R | ENST00000621329, | [P10636-8] |
| | ENSP00000477703, | |
| | ENSG00000276155 | |
| Tau D-0N4R | ENST00000622106, | [P10636-6] |
| | ENSP00000482244, | |
| | ENSG00000277956 | |
| Tau D-0N4R | ENST00000622728, | [P10636-6] |
| | ENSP00000479142, | |
| | ENSG00000276155 | |
| Tau D-0N4R | ENST00000626571, | [P10636-6] |
| | ENSP00000486039, | |
| | ENSG00000276155 | |
| fetal Tau 0N3R | ENST00000628393, | [P10636-2] |
| | ENSP00000487570, | |
| | ENSG00000276155 | |
| Tau C-2N3R | ENST00000631447, | [P10636-5] |
| | ENSP00000488373, | |
| | ENSG00000277956 | |
| Tau E-1N4R | ENST00000632500, | [P10636-7] |
| | ENSP00000487837, | |
| | ENSG00000277956 | |
| fetal Tau 0N3R | ENST00000633047, | [P10636-2] |
| | ENSP00000488245, | |
| | ENSG00000277956 | |
| Tau F-2N4R | ENST00000634049, | [P10636-8] |
| | ENSP00000487819, | |
| | ENSG00000277956 | |
| Tau E-1N4R | ENST00000680542, | [P10636-7] |
| | ENSP00000505258, | |
| | ENSG00000186868 | |

### REFERENCES

Avila, J., Lucas, J. J., Perez, M., and Hernandez, F. (2004). Role of tau protein in both physiological and pathological conditions. Physiol Rev 84, 361-384.
Benhelli-Mokrani, H., Mansuroglu, Z., Chauderlier, A., Albaud, B., Gentien, D., Sommer, S., Schirmer, C., Laqueuvre, L., Josse, T., Buee, L., Lefebvre, B., Galas, M. C., Soues, S., and Bonnefoy, E. (2018). Genome-wide identification of genic and intergenic neuronal DNA regions bound by Tau protein under physiological and stress conditions. Nucleic Acids Res 46, 11405-11422.
Bou Samra, E., Buhagiar-Labarchede, G., Machon, C., Guitton, J., Onclercq-Delic, R., Green, M. R., Alibert, O., Gazin, C., Veaute, X., and Amor-Gueret, M. (2017). A role for Tau protein in maintaining ribosomal DNA stability and cytidine deaminase-deficient cell survival. Nat Commun 8, 693.
Bryant, G. O. (2012). Measuring nucleosome occupancy in vivo by micrococcal nuclease. Methods Mol Biol 833, 47-61.
Bukar Maina, M., Al-Hilaly, Y. K., and Serpell, L. C. (2016). Nuclear Tau and Its Potential Role in Alzheimer's Disease. Biomolecules 6, 9.
Bustos, M. A., Salomon, M. P., Nelson, N., Hsu, S. C., Dinome, M. L., Hoon, D. S., and Marzese, D. M. (2017). Genome-wide chromatin accessibility, DNA methylation and gene expression analysis of histone deacetylase inhibition in triple-negative breast cancer. Genom Data 12, 14-16.
Chauderlier, A., Gilles, M., Spolcova, A., Caillierez, R., Chwastyniak, M., Kress, M., Drobecq, H., Bonnefoy, E., Pinet, F., Weil, D., Buee, L., Galas, M. C., and Lefebvre, B. (2018). Tau/DDX6 interaction increases microRNA activity. Biochim Biophys Acta Gene Regul Mech 1861, 762-772.
Cohen, T. J., Friedmann, D., Hwang, A. W., Marmorstein, R., and Lee, V. M. (2013). The microtubule-associated tau protein has intrinsic acetyltransferase activity. Nat Struct Mol Biol 20, 756-762.
Cowell, I. G., Papageorgiou, N., Padget, K., Watters, G. P., and Austin, C. A. (2011). Histone deacetylase inhibition redistributes topoisomerase Ilbeta from heterochromatin to euchromatin. Nucleus 2, 61-71.
Frost, B., Hemberg, M., Lewis, J., and Feany, M. B. (2014). Tau promotes neurodegeneration through global chromatin relaxation. Nat Neurosci 17, 357-366.
Galas, M. C., Dourlen, P., Begard, S., Ando, K., Blum, D., Hamdane, M., and Buee, L. (2006). The peptidylprolyl cis/trans-isomerase Pin1 modulates stress-induced dephosphorylation of Tau in neurons. Implication in a pathological mechanism related to Alzheimer disease. J Biol Chem 281, 19296-19304.
Gargini, R., Segura-Collar, B., and Sanchez-Gomez, P. (2019). Novel Functions of the Neurodegenerative-Related Gene Tau in Cancer. Front Aging Neurosci 11, 231.
Glaser, K. B., Staver, M. J., Waring, J. F., Stender, J., Ulrich, R. G., and Davidsen, S. K. (2003). Gene expression profiling of multiple histone deacetylase (HDAC) inhibitors: defining a common gene set produced by HDAC inhibition in T24 and MDA carcinoma cell lines. Mol Cancer Ther 2, 151-163.
Goedert, M., and Spillantini, M. G. (2000). Tau mutations in frontotemporal dementia FTDP-17 and their relevance for Alzheimer's disease. Biochim Biophys Acta 1502, 110-121.
Gorisch, S. M., Wachsmuth, M., Toth, K. F., Lichter, P., and Rippe, K. (2005). Histone acetylation increases chromatin accessibility. J Cell Sci 118, 5825-5834.
Henikoff, S., Henikoff, J. G., Sakai, A., Loeb, G. B., and Ahmad, K. (2009). Genome-wide profiling of salt fractions maps physical properties of chromatin. Genome Res 19, 460-469.
Hirose, T., Sowa, Y., Takahashi, S., Saito, S., Yasuda, C., Shindo, N., Furuichi, K., and Sakai, T. (2003). p53-independent induction of Gadd45 by histone deacetylase inhibitor: coordinate regulation by transcription factors Oct-1 and NF-Y. Oncogene 22, 7762-7773.
Itahana, Y., Zhang, J., Goke, J., Vardy, L. A., Han, R., Iwamoto, K., Cukuroglu, E., Robson, P., Pouladi, M. A., Colman, A., and Itahana, K. (2016). Histone modifications and p53 binding poise the p21 promoter for activation in human embryonic stem cells. Sci Rep 6, 28112.
Johnson, C. A., O'neill, L. P., Mitchell, A., and Turner, B. M. (1998). Distinctive patterns of histone H4 acetylation are associated with defined sequence elements within both heterochromatic and euchromatic regions of the human genome. Nucleic Acids Res 26, 994-1001.
Klein, H. U., Mccabe, C., Gjoneska, E., Sullivan, S. E., Kaskow, B. J., Tang, A., Smith, R. V., Xu, J., Pfenning, A. R., Bernstein, B. E., Meissner, A., Schneider, J. A., Mostafavi, S., Tsai, L. H., Young-Pearse, T. L., Bennett, D. A., and De Jager, P. L. (2019). Epigenome-wide study uncovers large-scale changes in histone acetylation driven by tau pathology in aging and Alzheimer's human brains. Nat Neurosci 22, 37-46.
Kreuz, S., and Fischle, W. (2016). Oxidative stress signaling to chromatin in health and disease. Epigenomics 8, 843-862.
Kubicek, S., O'sullivan, R. J., August, E. M., Hickey, E. R., Zhang, Q., Teodoro, M. L., Rea, S., Mechtler, K., Kowalski, J. A., Homon, C. A., Kelly, T. A., and Jenuwein, T. (2007). Reversal of H3K9me2 by a small-molecule inhibitor for the G9a histone methyltransferase. Mol Cell 25, 473-481.
Lebouvier, T., Pasquier, F., and Buee, L. (2017). Update on tauopathies. Curr Opin Neurol 30, 589-598.
Lefebvre, B., Brand, C., Flajollet, S., and Lefebvre, P. (2006). Down-regulation of the tumor suppressor gene retinoic acid receptor beta2 through the phosphoinositide 3-kinase/Akt signaling pathway. Mol Endocrinol 20, 2109-2121.
Lei, C., Yang, C., Xia, B., Ji, F., Zhang, Y., Gao, H., Xiong, Q., Lin, Y., Zhuang, X., Zhang, L., Zhu, T., Cheng, M., Yang, M., and Wang, K. (2020). Analysis of Tau Protein Expression in Predicting Pathological Complete Response to Neoadjuvant Chemotherapy in Different Molecular Subtypes of Breast Cancer. J Breast Cancer 23, 47-58.
Li, Y., and Seto, E. (2016). HDACs and HDAC Inhibitors in Cancer Development and Therapy. Cold Spring Harb Perspect Med 6.
Li, Z. H., Xiong, Q. Y., Tu, J. H., Gong, Y., Qiu, W., Zhang, H. Q., Wei, W. S., Hou, Y. F., and Cui, W. Q. (2013). Tau proteins expressions in advanced breast cancer and its significance in taxane-containing neoadjuvant chemotherapy. Med Oncol 30, 591.
Lippens, G., Wieruszeski, J. M., Leroy, A., Smet, C., Sillen, A., Buee, L., and Landrieu, I. (2004). Proline-directed random-coil chemical shift values as a tool for the NMR assignment of the tau phosphorylation sites. Chembiochem 5, 73-78.
Lu, M., and Kosik, K. S. (2001). Competition for microtubule-binding with dual expression of tau missense and splice isoforms. Mol Biol Cell 12, 171-184.
Mansuroglu, Z., Benhelli-Mokrani, H., Marcato, V., Sultan, A., Violet, M., Chauderlier, A., Delattre, L., Loyens, A., Talahari, S., Begard, S., Nesslany, F., Colin, M., Soues, S., Lefebvre, B., Buee, L., Galas, M. C., and Bonnefoy, E. (2016). Loss of Tau protein affects the structure, transcription and repair of neuronal pericentromeric heterochromatin. Sci Rep 6, 33047.
Marchion, D. C., Bicaku, E., Daud, A. I., Sullivan, D. M., and Munster, P. N. (2005). Valproic acid alters chromatin structure by regulation of chromatin modulation proteins. Cancer Res 65, 3815-3822.
Matrone, M. A., Whipple, R. A., Thompson, K., Cho, E. H., Vitolo, M. I., Balzer, E. M., Yoon, J. R., Ioffe, O. B., Tuttle, K. C., Tan, M., and Martin, S. S. (2010). Metastatic breast tumors express increased tau, which promotes microtentacle formation and the reattachment of detached breast tumor cells. Oncogene 29, 3217-3227.
Mishra, L. N., Pepenella, S., Rogge, R., Hansen, J. C., and Hayes, J. J. (2016). Acetylation Mimics Within a Single Nucleosome Alter Local DNA Accessibility In Compacted Nucleosome Arrays. Sci Rep 6, 34808.
Mitsiades, C. S., Mitsiades, N. S., Mcmullan, C. J., Poulaki, V., Shringarpure, R., Hideshima, T., Akiyama, M., Chauhan, D., Munshi, N., Gu, X., Bailey, C., Joseph, M., Libermann, T. A., Richon, V. M., Marks, P. A., and Anderson, K. C. (2004). Transcriptional signature of histone deacetylase inhibition in multiple myeloma: biological and clinical implications. Proc Natl Acad Sci USA 101, 540-545.
Morgan, M. A., and Shilatifard, A. (2015). Chromatin signatures of cancer. Genes Dev 29, 238-249.
Newbold, A., Salmon, J. M., Martin, B. P., Stanley, K., and Johnstone, R. W. (2014). The role of p21(waf1/cip1) and p27(Kip1) in HDACi-mediated tumor cell death and cell cycle arrest in the Emu-myc model of B-cell lymphoma. Oncogene 33, 5415-5423.
Peart, M. J., Smyth, G. K., Van Laar, R. K., Bowtell, D. D., Richon, V. M., Marks, P. A., Holloway, A. J., and Johnstone, R. W. (2005). Identification and functional significance of genes regulated by structurally different histone deacetylase inhibitors. Proc Natl Acad Sci USA 102, 3697-3702.
Qi, H., Cantrelle, F. X., Benhelli-Mokrani, H., Smet-Nocca, C., Buee, L., Lippens, G., Bonnefoy, E., Galas, M. C., and Landrieu, I. (2015). Nuclear magnetic resonance spectroscopy characterization of interaction of Tau with DNA and its regulation by phosphorylation. Biochemistry 54, 1525-1533.
Richon, V. M., Sandhoff, T. W., Rifkind, R. A., and Marks, P. A. (2000). Histone deacetylase inhibitor selectively induces p21WAF1 expression and gene-associated histone acetylation. Proc Natl Acad Sci USA 97, 10014-10019.
Robbins, A. R., Jablonski, S. A., Yen, T. J., Yoda, K., Robey, R., Bates, S. E., and Sackett, D. L. (2005). Inhibitors of histone deacetylases alter kinetochore assembly by disrupting pericentromeric heterochromatin. Cell Cycle 4, 717-726.
Rossi, G., Redaelli, V., Perego, P., Ferrari, R., Giaccone, G., and Tagliavini, F. (2018). Tau Mutations as a Novel Risk Factor for Cancer-Response. Cancer Res 78, 6525.
Rouzier, R., Rajan, R., Wagner, P., Hess, K. R., Gold, D. L., Stec, J., Ayers, M., Ross, J. S., Zhang, P., Buchholz, T. A., Kuerer, H., Green, M., Arun, B., Hortobagyi, G. N., Symmans, W. F., and Pusztai, L. (2005). Microtubule-associated protein tau: a marker of paclitaxel sensitivity in breast cancer. Proc Natl Acad Sci USA 102, 8315-8320.
Sergeant, N., Delacourte, A., and Buee, L. (2005). Tau protein as a differential biomarker of tauopathies. Biochim Biophys Acta 1739, 179-197.
Shogren-Knaak, M., Ishii, H., Sun, J. M., Pazin, M. J., Davie, J. R., and Peterson, C. L. (2006). Histone H4-K16 acetylation controls chromatin structure and protein interactions. Science 311, 844-847.
Siano, G., Caiazza, M. C., Olla, I., Varisco, M., Madaro, G., Quercioli, V., Calvello, M., Cattaneo, A., and Di Primio, C. (2019). Identification of an ERK Inhibitor as a Therapeutic Drug Against Tau Aggregation in a New Cell-Based Assay. Front Cell Neurosci 13, 386.
Smoter, M., Bodnar, L., Duchnowska, R., Stec, R., Grala, B., and Szczylik, C. (2011). The role of Tau protein in resistance to paclitaxel. Cancer Chemother Pharmacol 68, 553-557.
Sotiropoulos, I., Galas, M. C., Silva, J. M., Skoulakis, E., Wegmann, S., Maina, M. B., Blum, D., Sayas, C. L., Mandelkow, E. M., Mandelkow, E., Spillantini, M. G., Sousa, N., Avila, J., Medina, M., Mudher, A., and Buee, L. (2017). Atypical, non-standard functions of the microtubule associated Tau protein. Acta Neuropathol Commun 5, 91.
Spicakova, T., O'brien, M. M., Duran, G. E., Sweet-Cordero, A., and Sikic, B. I. (2010). Expression and silencing of the microtubule-associated protein Tau in breast cancer cells. Mol Cancer Ther 9, 2970-2981.
Taddei, A., Maison, C., Roche, D., and Almouzni, G. (2001). Reversible disruption of pericentric heterochromatin and centromere function by inhibiting deacetylases. Nat Cell Biol 3, 114-120.
Takekawa, M., and Saito, H. (1998). A family of stress-inducible GADD45-like proteins mediate activation of the stress-responsive MTK1/MEKK4 MAPKKK. Cell 95, 521-530.
Taylor, G. C., Eskeland, R., Hekimoglu-Balkan, B., Pradeepa, M. M., and Bickmore, W. A. (2013). H4K16 acetylation marks active genes and enhancers of embryonic stem cells, but does not alter chromatin compaction. Genome Res 23, 2053-2065.
Toth, K. F., Knoch, T. A., Wachsmuth, M., Frank-Stohr, M., Stohr, M., Bacher, C. P., Muller, G., and Rippe, K. (2004). Trichostatin A-induced histone acetylation causes decondensation of interphase chromatin. J Cell Sci 117, 4277-4287.
Turner, B. M., Birley, A. J., and Lavender, J. (1992). Histone H4 isoforms acetylated at specific lysine residues define individual chromosomes and chromatin domains in Drosophila polytene nuclei. Cell 69, 375-384.
Wagner, P., Wang, B., Clark, E., Lee, H., Rouzier, R., and Pusztai, L. (2005). Microtubule Associated Protein (MAP)-Tau: a novel mediator of paclitaxel sensitivity in vitro and in vivo. Cell Cycle 4, 1149-1152.
Wang, Y., and Mandelkow, E. (2016). Tau in physiology and pathology. Nat Rev Neurosci 17, 5-21.
Yu, Q., Olsen, L., Zhang, X., Boeke, J. D., and Bi, X. (2011). Differential contributions of histone H3 and H4 residues to heterochromatin structure. Genetics 188, 291-308.
Zhang, W., Hoffman, B., and Liebermann, D. A. (2001). Ectopic expression of MyD118/Gadd45/CR6 (Gadd45beta/alpha/gamma) sensitizes neoplastic cells to genotoxic stress-induced apoptosis. Int J Oncol 18, 749-757.

1. A method of treating cancer in a patient undergoing histone deacetylase (HDAC) inhibitor therapy, comprising the steps of:
determining in a biological sample obtained from said patient a Tau expression level, and
administering a therapeutically effective amount of an HDAC inhibitor to the patient when the Tau expression level is low or null.

2. A method of preventing emergence of resistance to treatment with a HDAC inhibitor in a subject in need thereof comprising administering to the subject, a Tau inhibitor.

3. A method for preventing and/or treating cancer with acquired resistance to treatment with a HDAC inhibitor in a subject in need thereof comprising administering to the subject a combination of drugs selected from the group consisting of an HDAC inhibitor and a Tau inhibitor

4. The method according to claim 1, wherein the HDAC inhibitor is selected from the group consisting of belinostat (PXD-101), vorinostat (SAHA), entinostat (MS-275) panabinostat (LBH-589), mocetinostat (MGCD0103), chidamide (HBI-8000) romidepsin (FK-228) and Trichostatin A (TSA).

5. The method according to claim 2 or 3, wherein the Tau inhibitor is
A) an inhibitor of Tau activity selected from the group consisting of an anti-Tau antibody, an anti-Tau aptamer, and a tau peptide,
B) Proteolysis Targeting Chimera (PROTAC) which simultaneously binds a tau protein and an E3-ubiquitin ligase or
C) an inhibitor of Tau gene expression selected from the group consisting of an antisense oligonucleotide, an oligonucleotide, a nuclease, an siRNA, an shRNA or a ribozyme nucleic acid sequence.

6. The method according to claim 5, wherein the tau Inhibitor is an anti-Tau antibody.

7. (canceled)

8. The method of according to claim 1, wherein the cancer is breast, gastric, prostate or ovarian cancer.
